(19) United States
(12) Patent Application Publication
(10) Pub. No.: US 2024/0229124 A9
(43) Pub. Date: Jul. 11, 2024

(54) OPTIMIZED CLUSTERING OF DNA STRANDS

(71) Applicant: DNAalgo S.r.l., Macerata (IT)

(72) Inventors: Thomas Chiozzi, Mantovano (IT); Lorenzo Zuolo, Lusia (IT); Alessia Marelli, Treviolo (IT); Rino Micheloni, Moltrasio (IT)

(73) Assignee: DNAalgo S.r.l., Macerata (IT)

(21) Appl. No.: 18/047,966

(22) Filed: Oct. 19, 2022

(51) Int. Cl. C12Q 1/6869; C12Q 1/6844

(52) U.S. Cl. CPC C12Q 1/6869; C12Q 1/6844

(57) ABSTRACT

A method for clustering nucleotide strings of DNA strand replicas in clusters, comprising, for each pair of a first nucleotide string and a second nucleotide string, carrying out: arranging a matrix wherein each matrix element corresponds to a selected nucleotide in the first nucleotide string and to a further selected nucleotide in the second nucleotide string and is configured to store a calculated edit value indicative of an edit distance; progressively filling the matrix by storing calculated edit values; if the edit value calculated for a matrix element belonging to an output diagonal of the matrix is not lower than a cluster threshold, stopping said progressively filling and placing said first and second nucleotide strings in two different clusters, said output diagonal comprising the matrix element corresponding to the last column and the last row of the matrix.

### BACKGROUND OF THE INVENTION

### Field of the Invention

The present invention generally relates to the field of data storage, and more specifically to the field of DNA data storage.

### Overview of the Related Art

DNA data storage is a technology for storing data in synthetic DNA, i.e., using molecules of synthetic DNA as a data storage medium. Compared to current data storage technologies, DNA data storage provides a largely improved data storage density and an improved durability.

As it is known, DNA consists of double stranded polymers of a set of four nucleotides: adenine (A), cytosine (C), guanine (G) and thymine (T). DNA data storage provides for encoding and decoding binary data to and from synthesized DNA strands.

Words of bits comprising 1 and 0 digit are encoded to nucleotide strings comprising a sequence of symbols each corresponding to a nucleotide and then corresponding artificial DNA strands are synthesized to comprise said nucleotide strings.

In order to read a group of DNA strands for retrieving the nucleotide strings therefrom to be decoded for obtaining the corresponding digital words, the DNA strands of the group are subjected to a sequencing procedure. The sequencing procedure provides for amplifying the DNA strands of the groups by generating a number of replicas of each DNA strand of the group. The DNA strands of the group to be read are also referred to as “reference DNA strands”, while the replicas thereof generated by the sequencing procedure are also referred to as “DNA strand replicas” or “DNA reads”.

The sequencing procedure carried out with current technologies has several limitations, capable of negatively affecting the correct outcome of the reading.

Indeed, according to the current sequencing technologies, the reference DNA strands are sequenced in an unordered manner, each reference DNA strand may be sequenced more than once, and the obtained DNA strand replicas may be affected by errors that may alter the content thereof with respect to the original reference DNA strands. For example, compared to a reference DNA strand of the group, the corresponding DNA strand replicas thereof may:
comprise one or more additional nucleotides not present in the reference DNA strand,
lack of one or more of the nucleotides of the reference DNA strand,
have some of the nucleotides that are ordered differently from the ones of the original DNA strand, and/or
may be mistaken for a different reference DNA strand of the group.

By making reference to the very simplified exemplary case illustrated in FIG. 1, a group 100 of four reference DNA strands RS(l) (l=1, . . . , 4) to be read is processed according to a sequencing procedure that produced seven DNA strands replicas RR(m) (m=1, . . . , 7).

The reference DNA strand RS(1) corresponds to the nucleotide string “AACGTAG”, the reference DNA strand RS(2) corresponds to the nucleotide string “GGTAGG”, the reference DNA strand RS(3) corresponds to the nucleotide string “CTTGATA” and the reference DNA strand RS(4) corresponds to the nucleotide string “TTAAGGC”.

The DNA strand replica RR(1) corresponds to the nucleotide string “CATGATA”, the DNA strand replica RR(2) corresponds to the nucleotide string “TTAAGGC”, the DNA strand replica RR(3) corresponds to the nucleotide string “AACTTAG”, the DNA strand replica RR(4) corresponds to the nucleotide string “AACTAG”, the DNA strand replica RR(5) corresponds to the nucleotide string “TTATAGGC”, the DNA strand replica RR(6) corresponds to the nucleotide string “GGTAG”, the DNA strand replica RR(7) corresponds to the nucleotide string “GGTAGG”.

From the example above, it can be understood that reading the actual data content of the reference DNA strands RS(1) of the group 100 is a not trivial task because of the noise introduced in the DNA strand replicas RR(m) by the sequencing procedure. Indeed, by observing the DNA strand replicas RR(m) of the example at issue:
the number of DNA strand replicas RR(m) generated for each reference DNA strand RS(1) is generally variable (for example, two strand replicas RR(3), RR(4), are generated from the reference DNA strand RS(1), while only one strand replica RR(1) is generated from the reference DNA strand RS(3));
the order of the DNA strand replicas RR(m) does not follow the order of the reference DNA strand RS(1) of the group 100 (for example, while the first two reference DNA strands RS(1) of the group 100 are RS(1), RS(2), the first two retrieved DNA strand replicas RR(1), RR(2) have been generated from the reference DNA strands RS(3), RS(4), respectively);
the nucleotide string of each DNA strand replica RR(m) may be generally affected by errors causing a DNA strand replica RR(m) to differ from the corresponding reference DNA strand RS(1) by insertions, deletions, and/or substitutions of nucleotides (for example, the DNA strand replica RR(4) differs from the corresponding reference DNA strand RS(1) because of the deletion of nucleotide G after nucleotide C, the DNA strand replica RR(5) differs from the corresponding reference DNA strand RS(4) because of the insertion of nucleotide T between the nucleotides A of the nucleotide substring “AA”, the DNA strand replica RR(1) differs from the corresponding reference DNA strand RS(3) because the nucleotide T in the nucleotide substring “CT” has been substituted with nucleotide A).

In order to carry out a correct reading of the reference DNA strands RS(l) of the group 100 from the DNA strand replicas RR(m) generated through the sequencing procedure, the nucleotide strings of the DNA strand replicas RR(m) are clustered into corresponding clusters C(p) (p=1, 2, . . . ), wherein each cluster C(p) comprises nucleotide strings of DNA strand replicas RR(m) that are similar to each other. Since each DNA strand replica RR(m) is a (possibly, noisy) replica of a corresponding reference DNA strand RS(l) of the group 100, and therefore the nucleotide string of the former will be similar to the nucleotide of the latter, there is a high probability that each of at least a subset of the clusters C(p) will generally comprise nucleotide strings of DNA strand replicas RR(m) that are similar to the nucleotide string of a corresponding reference DNA strand RS(1) of the group 100.

By making reference to FIG. 2, in the example at issue, four clusters C(p) are generated and namely:
a first cluster C(1), comprising the nucleotide strings of the DNA strand replicas RR(3) and RR(4);
a second cluster C(2), comprising the nucleotide strings of the DNA strand replicas RR(6) and RR(7);
a third cluster C(3), comprising the nucleotide string of the DNA strand replica RR(1);
a fourth cluster C(4), comprising the nucleotide strings of the DNA strand replicas RR(2) and RR(5).

The cluster C(1) comprises nucleotide strings of DNA strand replicas that are similar to the nucleotide string of the reference DNA strand RS(1), the cluster C(2) comprises nucleotide strings of DNA strand replicas that are similar to the nucleotide string of the reference DNA strand RS(2), the cluster C(p) comprises a nucleotide string of a DNA strand replica that is similar to the nucleotide string of the reference DNA strand RS(3), the cluster C(4) comprises nucleotide strings of DNA strand replicas that are similar to the nucleotide string of the reference DNA strand RS(4).

The clusters C(p) comprising the nucleotide strings of the DNA strand replicas RR(m) are then processed to identify for each cluster C(p) which reference DNA strand RS(l) said cluster C(p) corresponds to, retrieving the data stored in the reference DNA strand RS(l) of the group 100. For example, the clusters C(p) may be processed according to a consensus-finding algorithm configured to predict the most likely reference DNA strand RS(l) to have produced the DNA strand replicas RR(m) of each cluster C(p).

The higher the precision of the clustering operations, the higher the reliability of the resulting reading outcome.

Known methods exist to quantify the “similarity” between the nucleotide strings of DNA strand replicas RR(m) for generating the clusters C(p), one of which provides for calculating the known edit distance ED, also referred to as “Levenshtein distance”.

The edit distance ED between two strings is defined as the minimum number of operations required to transform one string into the other. In other words, by making reference to the case at issue, in which the nucleotide strings corresponding to the DNA strand replicas RR(m) comprise a sequence of symbols taken from the alphabet {A, C, G, T}, the edit distance ED between a pair DNA strand replicas RR(m) is the minimum number of symbols (nucleotides) to be included/removed/replaced into/from the nucleotide string of a DNA strand replica RR(m) of the pair to obtain the nucleotide string of the other DNA strand replica RR(m).

For example, the edit distance ED between the nucleotide strings of the DNA strand replicas RR(6) (“GGTAG”) and RR(7) (“GGTAGG”) is equal to one, because it is sufficient to carry out a single operation to modify the nucleotide string of RR(6) into the nucleotide string of RR(7) (i.e., removing the last nucleotide G) or to modify the nucleotide string of RR(7) into the nucleotide string of RR(6) (i.e., adding a nucleotide G after the last nucleotide G).

Two nucleotide strings of DNA strand replicas RR(m) are clustered into a same corresponding cluster C(p) if the corresponding edit distance ED is lower than a corresponding cluster threshold TH, otherwise they are clustered in different clusters.

A known method for calculating the edit distance ED between two strings is the so-called Wagner-Fischer algorithm—also known as Needleman-Wunsch algorithm or Scott-Waterman algorithm—(Robert A. Wagner and Michael J. Fischer, 1974, “The String-to-String Correction Problem” J. ACM 21, 1 (January 1974), 168-173, DOI: 10.1145/321796.321811).

With reference to FIGS. 3A-3D, an example will be now described of how the edit distance ED between a pair of a first and a second nucleotide strings of a respective first and second DNA strand replicas RR(m) is calculated according to the Wagner-Fischer algorithm.

The algorithm provides for arranging a matrix D having N+1 rows and M+1 columns, wherein N is the number of symbols (nucleotides) in the nucleotide string of the first DNA strand replica RR(m) and M is the number of symbols (nucleotides) in the nucleotide string of the second DNA strand replica RR(m). By considering an example in which the first DNA strand replica is RR(7) (corresponding to the nucleotide string “GGTAGG”) and the second DNA strand replica is RR(6) (corresponding to the nucleotide string “GGTAG”), N=6 and M=5.

By identifying:
the rows of the matrix D with r(i) (i=0 to N),
the columns of the matrix D with c(j) (j=0 to M),
the i-th nucleotide of the nucleotide string of the first DNA strand replica RR(m) with x(i),
the j-th nucleotide of the nucleotide string of the second DNA strand replica RR(m) with y(j),
   the elements d[0, j] (j=0 to M) of the row r(0) are initialized to values ED(0,j)=j, respectively, the elements d[i, 0] (i=0 to N) of the column c(0) are initialized to values ED(i,0)=i, each one of the N rows r(i) (i=1 to N) of the matrix D is associated to a corresponding nucleotide x(i), and each one of the M columns c(j) (j=1 to M) of the matrix D is associated to a corresponding nucleotide y(j) (see FIG. 3A).

The generic matrix element d[i,j] (i=1 to N, j=1 to M) corresponds to the nucleotide x(i) of the nucleotide string of the first DNA strand replica RR(m) and to the nucleotide y(j) of the nucleotide string of the second DNA strand replica RR(m), and is configured to store a value of a calculated edit distance ED(i,j) between a prefix of the nucleotide string of the first DNA strand replica RR(m) ending with the nucleotide x(i) and a prefix of the nucleotide string of the second DNA strand replica RR(m) ending with the nucleotide y(i). The matrix element d[N,M] corresponding to the last row r(N) and last column c(M) is thus configured to store the edit distance ED(N,M)=ED between the entire nucleotide string of the first DNA strand replica RR(m) and the entire nucleotide string of the second DNA strand replica RR(m).

By making reference to the case at issue, the matrix element d[2,4] is configured to store the edit distance ED(2,4) between the substring “GG” of the DNA strand replica RR(7), and the substring “GGTA” of the DNA strand replica RR(6), and the matrix element d[N=6,M=5] is configured to store the edit distance ED between the entire nucleotide string of the first DNA strand replica RR(7) and the entire nucleotide string of the second DNA strand replica RR(6).

The edit distances ED(i,j) to be stored in the matrix elements d[i,j] (i=1 to N, j=1 to M) are recursively calculated, starting from the matrix element d[1,1], in the following way:

$$ED ( i , j ) = min ( \begin{matrix}ED ( i - 1 , j - 1 ) + s \\ ED ( i - 1 , j ) + 1 \\ ED ( i , j - 1 ) + 1\end{matrix} )$$

$$wherein :$$

$$s = \{ \begin{matrix}0 & if x ( i ) = y ( j ) \\ 1 & if x ( i ) \neq y ( j )\end{matrix}$$

The calculation of the edit distances ED(i,j) (and the corresponding storage thereof in the matrix elements d[i,j]) may be carried out progressively, for example by proceeding row-by-row or column-by-column, until the last edit distance ED(N,M)=ED between the two entire nucleotide strings of the two DNA strand replicas RR(m) is calculated.

By making reference to the case at issue, and by proceeding row-by-row, the edit distance ED(1,1) to be stored in the matrix element d[1,1] is equal to

$$min ( \begin{matrix}0 + 0 \\ 1 + 1 \\ 1 + 1\end{matrix} ) = 0$$

(see FIG. 3B), the edit distance ED(1,2) to be stored in the matrix element d[1,2] is equal to

$$min ( \begin{matrix}0 + 1 \\ 2 + 1 \\ 0 + 1\end{matrix} ) = 1$$

(see FIG. 3C), and so on, until calculating the last edit distance ED(N,M)=ED between the entire nucleotide strings of the DNA strand replicas RR(7) and RR(6) to be stored in the matrix element d[6,5], which is equal to

$$min ( \begin{matrix}1 + 0 \\ 0 + 1 \\ 2 + 1\end{matrix} ) = 1 ,$$

as shown in FIG. 3D.

### SUMMARY OF THE INVENTION

The Applicant has recognized that the known solutions currently employed for clustering nucleotide strings of DNA strand replicas for reading synthetic DNA strands are not efficient.

Indeed, in a practical scenario, the nucleotide strings of synthetic DNA strands to be read are long, including sequences of hundreds or thousands of nucleotides, and the clustering procedure provides for clustering billions of nucleotide strings.

Carrying out the operations described above for calculating edit distances ED for a so large number of nucleotide strings comprising a very large number of nucleotides involves the generation of large numbers of large matrixes and the calculation of a corresponding extremely large number of operations for filling each matrix element, dramatically increasing the costs in terms of time, computational load and electric power.

The Applicant has tackled the above-discussed issues, and has devised an improved solution for clustering nucleotide strings of DNA requiring a reduced number of operations, reducing thus the costs in terms of time, computational load and electric power.

One or more aspects of the present invention are set out in the independent claims, with advantageous features of the same invention that are indicated in the dependent claims, whose wording is enclosed herein verbatim by reference (with any advantageous feature being provided with reference to a specific aspect of the present invention that applies mutatis mutandis to any other aspect thereof).

More specifically, an aspect of the present invention relates to a method for reading a group of synthetic DNA strands.

The method comprises amplifying the DNA strands of the group so as to generate, for each DNA strand of the group, at least a corresponding DNA strand replica.

Each DNA strand replica corresponds to a respective nucleotide string comprising a sequence of nucleotides of the DNA strand replica.

The method further comprises clustering the nucleotide strings of the generated DNA strand replicas in respective clusters so that each cluster comprises nucleotide strings having edit distances among them that are lower than a cluster threshold.

The method further comprises obtaining a reading of at least one DNA strand of the group based on the nucleotide strings comprised in a at least one among said clusters.

Said clustering the nucleotide strings in respective clusters comprises:
for each pair of a first nucleotide string and a second nucleotide string, carrying out the following sequence of operations:
   arranging a matrix of matrix elements, the matrix comprising a respective row for each nucleotide in the first nucleotide string, and a respective column for each nucleotide in the second nucleotide string, each matrix element which corresponds to the row corresponding to a selected nucleotide in the first nucleotide string and to the column corresponding to a further selected nucleotide in the second nucleotide string being configured to store a calculated edit value indicative of an edit distance between a prefix of the first nucleotide string ending with said selected nucleotide and a prefix of the second nucleotide string ending with said selected further nucleotide;
   starting from the matrix element which corresponds to the row corresponding to the first nucleotide of the first nucleotide string and to the column corresponding to the first nucleotide of the second nucleotide string, progressively filling a group of matrix elements by storing calculated edit values indicative of edit distances corresponding to said matrix elements;
   if the edit value calculated for a matrix element belonging to an output diagonal of the matrix is not lower than the cluster threshold, stopping said progressively filling the group of matrix elements and placing said first nucleotide string and said second nucleotide string in two different clusters, said output diagonal being a diagonal of the matrix comprising the matrix element corresponding to the last column and the last row of the matrix.

According to an embodiment of the present invention, the method further comprises:
if the edit value calculated for the matrix element corresponding to the last column and the last row of the matrix is lower than the cluster threshold, placing said first nucleotide string and said second nucleotide string in a same cluster.

According to an embodiment of the present invention, said filling a group of matrix elements by storing calculated edit values comprises storing into a selected matrix element corresponding to a selected row and to a selected column a calculated edit value that is calculated based on a comparison between:
the nucleotide in the first nucleotide string corresponding to said selected row, and
the nucleotide in the second nucleotide string corresponding to said selected column.

According to an embodiment of the present invention, said filling the matrix elements by storing calculated edit values comprises storing into a selected matrix element corresponding to a selected row and to a selected column a calculated edit value that is calculated based on a comparison among already calculated edit values stored in matrix elements adjacent to said selected matrix element.

According to an embodiment of the present invention, said filling the matrix elements by storing calculated edit values comprises storing into a selected matrix element corresponding to a selected row and to a selected column a calculated edit value that is calculated according to the following procedure:
setting a parameter to zero if the nucleotide in the first nucleotide string corresponding to said selected row is equal to the nucleotide in the second nucleotide string corresponding to said selected column;
setting said parameter to one if the nucleotide in the first nucleotide string corresponding to said selected row is different from the nucleotide in the second nucleotide string corresponding to said selected column;
setting the edit value as the minimum among a), b), c):
a) the value of said parameter plus the calculated edit value stored in the matrix element corresponding to:
   the row of the matrix adjacent to and preceding the selected row, and
   the column of the matrix adjacent to and preceding the selected column;
b) one plus the calculated edit value stored in the matrix element corresponding to:
   the row of the matrix adjacent to and preceding the selected row, and
   the selected column;
c) one plus the calculated edit value stored in the matrix element corresponding to:
   the selected row, and
   the column of the matrix adjacent to and preceding the selected column.

According to an embodiment of the present invention, the rows of the matrix are ordered according to the order of the nucleotides in the first nucleotide string.

According to an embodiment of the present invention, the columns of the matrix are ordered according to the order of the nucleotides in the second nucleotide string.

According to an embodiment of the present invention, said matrix further comprises:
an initialization row adjacent to and preceding the row corresponding to the first nucleotide of the first nucleotide string;
an initialization column adjacent to and preceding the column corresponding to the second nucleotide of the second nucleotide string.

According to an embodiment of the present invention, the method further comprises:
before said progressively filling the group of matrix elements starting from the matrix element which corresponds to the row corresponding to the first nucleotide of the first nucleotide string and to the column corresponding to the first nucleotide of the second nucleotide string, carrying out the following operations:
initializing the matrix elements of the initialization row by storing edit values having an increasing value, starting from zero, and progressively increasing by one at each adjacent subsequent matrix element of the initialization row;
initializing the matrix values of the initialization column by storing edit values having an increasing value, starting from zero, and progressively increasing by one at each adjacent subsequent matrix element of the initialization column.

According to an embodiment of the present invention, said progressively filling the group of matrix elements by storing calculated edit values comprises calculating and storing edit values by proceeding according to a row-by-row pattern starting from said matrix element which corresponds to the row corresponding to the first nucleotide of the first nucleotide string and to the column corresponding to the first nucleotide of the second nucleotide string.

According to an embodiment of the present invention, said progressively filling the group of matrix elements by storing calculated edit values comprises calculating and storing edit values by proceeding according to a column-by-column pattern starting from said matrix element which corresponds to the row corresponding to the first nucleotide of the first nucleotide string and to the column corresponding to the first nucleotide of the second nucleotide string.

According to an embodiment of the present invention, said progressively filling the group of matrix elements by storing calculated edit values comprises calculating and storing edit values by proceeding according to an antidiagonal-by-antidiagonal pattern starting from said matrix element which corresponds to the row corresponding to the first nucleotide of the first nucleotide string and to the column corresponding to the first nucleotide of the second nucleotide string.

According to an embodiment of the present invention, said group of matrix elements comprise all the matrix elements of the matrix.

According to an embodiment of the present invention, said group of matrix elements comprise the matrix elements corresponding to the output diagonal.

According to an embodiment of the present invention, said group of matrix elements comprise matrix elements comprised between two diagonals of the matrix different from the output diagonal, the output diagonal falling between said two diagonals.

According to an embodiment of the present invention, said two diagonals of the matrix different from the output diagonal comprise:
a first diagonal that is displaced with respect to the output diagonal by a corresponding number of matrix elements;
a second diagonal that is displaced with respect to the output diagonal by said number of matrix elements.

According to an embodiment of the present invention, said two diagonals of the matrix different from the output diagonal comprise:
a first diagonal that is displaced with respect to the main diagonal of the matrix by a corresponding number of matrix elements;
a second diagonal that is displaced with respect to the main diagonal of the matrix by said number of matrix elements.

According to an embodiment of the present invention, said two diagonals of the matrix different from the output diagonal comprise:
a first diagonal that is displaced with respect to the main diagonal of the matrix by a corresponding number of matrix elements;
a second diagonal that is displaced with respect to the output diagonal by said number of matrix elements.

Another aspect of the present invention relates to a method for clustering a group of synthetic DNA strands, each one corresponding to a respective nucleotide string comprising a sequence of nucleotides of the DNA strand, in respective clusters so that each cluster comprises nucleotide strings having edit distances among them that are lower than a cluster threshold.

The method comprises for each pair of a first nucleotide string and a second nucleotide string, carrying out the following sequence of operations:
arranging a matrix of matrix elements, the matrix comprising a respective row for each nucleotide in the first nucleotide string, and a respective column for each nucleotide in the second nucleotide string, each matrix element which corresponds to the row corresponding to a selected nucleotide in the first nucleotide string and to the column corresponding to a further selected nucleotide in the second nucleotide string being configured to store a calculated edit value indicative of an edit distance between a prefix of the first nucleotide string ending with said selected nucleotide and a prefix of the second nucleotide string ending with said selected further nucleotide;
starting from the matrix element which corresponds to the row corresponding to the first nucleotide of the first nucleotide string and to the column corresponding to the first nucleotide of the second nucleotide string, progressively filling a group of matrix elements by storing calculated edit values indicative of edit distances corresponding to said matrix elements;
if the edit value calculated for a matrix element belonging to an output diagonal of the matrix is not lower than the cluster threshold, stopping said progressively filling the group of matrix elements and placing said first nucleotide string and said second nucleotide string in two different clusters, said output diagonal being a diagonal of the matrix comprising the matrix element corresponding to the last column and the last row of the matrix.

According to an embodiment of the present invention, the method further comprises:
if the edit value calculated for the matrix element corresponding to the last column and the last row of the matrix is lower than the cluster threshold, placing said first nucleotide string and said second nucleotide string in a same cluster.

According to an embodiment of the present invention, said filling the matrix elements by storing calculated edit values comprises storing into a selected matrix element corresponding to a selected row and to a selected column a calculated edit value that is calculated according to the following procedure:
setting a parameter to zero if the nucleotide in the first nucleotide string corresponding to said selected row is equal to the nucleotide in the second nucleotide string corresponding to said selected column;
setting said parameter to one if the nucleotide in the first nucleotide string corresponding to said selected row is different from the nucleotide in the second nucleotide string corresponding to said selected column;
setting the edit value as the minimum among a), b), c):
a) the value of said parameter plus the calculated edit value stored in the matrix element corresponding to:
   the row of the matrix adjacent to and preceding the selected row, and
   the column of the matrix adjacent to and preceding the selected column;
b) one plus the calculated edit value stored in the matrix element corresponding to:
   the row of the matrix adjacent to and preceding the selected row, and
   the selected column;
c) one plus the calculated edit value stored in the matrix element corresponding to:
   the selected row, and
   the column of the matrix adjacent to and preceding the selected column.

According to an embodiment of the present invention:
the rows of the matrix are ordered according to the order of the nucleotides in the first nucleotide string;
the columns of the matrix are ordered according to the order of the nucleotides in the second nucleotide string.

According to an embodiment of the present invention, said matrix further comprises:
an initialization row adjacent to and preceding the row corresponding to the first nucleotide of the first nucleotide string;
an initialization column adjacent to and preceding the column corresponding to the second nucleotide of the second nucleotide string.

According to an embodiment of the present invention, the method further comprises:
before said progressively filling the group of matrix elements starting from the matrix element which corresponds to the row corresponding to the first nucleotide of the first nucleotide string and to the column corresponding to the first nucleotide of the second nucleotide string, carrying out the following operations:
initializing the matrix elements of the initialization row by storing edit values having an increasing value, starting from zero, and progressively increasing by one at each adjacent subsequent matrix element of the initialization row;
initializing the matrix values of the initialization column by storing edit values having an increasing value, starting from zero, and progressively increasing by one at each adjacent subsequent matrix element of the initialization column.

According to an embodiment of the present invention, said progressively filling the group of matrix elements by storing calculated edit values comprises calculating and storing edit values by proceeding according to a row-by-row pattern starting from said matrix element which corresponds to the row corresponding to the first nucleotide of the first nucleotide string and to the column corresponding to the first nucleotide of the second nucleotide string.

According to an embodiment of the present invention, said progressively filling the group of matrix elements by storing calculated edit values comprises calculating and storing edit values by proceeding according to a column-by-column pattern starting from said matrix element which corresponds to the row corresponding to the first nucleotide of the first nucleotide string and to the column corresponding to the first nucleotide of the second nucleotide string.

According to an embodiment of the present invention, said progressively filling the group of matrix elements by storing calculated edit values comprises calculating and storing edit values by proceeding according to an antidiagonal-by-antidiagonal pattern starting from said matrix element which corresponds to the row corresponding to the first nucleotide of the first nucleotide string and to the column corresponding to the first nucleotide of the second nucleotide string.

According to an embodiment of the present invention, said group of matrix elements comprise the matrix elements corresponding to the output diagonal.

According to an embodiment of the present invention, said group of matrix elements comprise matrix elements comprised between two diagonals of the matrix different from the output diagonal, the output diagonal falling between said two diagonals.

Another aspect of the present invention relates to a synthetic DNA storage system.

The synthetic DNA storage system comprises a DNA storage module configured to store synthetic DNA strands.

The synthetic DNA storage system comprises a sequencer module configured to amplify a group of stored synthetic DNA strands so as to generate, for each DNA strand of the group, at least a corresponding DNA strand replica, each DNA strand replica corresponding to a respective nucleotide string comprising a sequence of nucleotides of the DNA strand replica.

The synthetic DNA storage system comprises a reading module configured to:
cluster the nucleotide strings of the generated DNA strand replicas in respective clusters so that each cluster comprises nucleotide strings having edit distances among them that are lower than a cluster threshold, and
obtain a reading of at least one DNA strand of the group based on the nucleotide strings comprised in a at least one among said clusters.

Said reading module is configured to cluster the nucleotide strings in respective clusters by carrying out for each pair of a first nucleotide string and a second nucleotide string the following sequence of operations:
arranging a matrix of matrix elements, the matrix comprising a respective row for each nucleotide in the first nucleotide string, and a respective column for each nucleotide in the second nucleotide string, each matrix element which corresponds to the row corresponding to a selected nucleotide in the first nucleotide string and to the column corresponding to a further selected nucleotide in the second nucleotide string being configured to store a calculated edit value indicative of an edit distance between a prefix of the first nucleotide string ending with said selected nucleotide and a prefix of the second nucleotide string ending with said selected further nucleotide;
starting from the matrix element which corresponds to the row corresponding to the first nucleotide of the first nucleotide string and to the column corresponding to the first nucleotide of the second nucleotide string, progressively filling a group of matrix elements by storing calculated edit values indicative of edit distances corresponding to said matrix elements;
if the edit value calculated for a matrix element belonging to an output diagonal of the matrix is not lower than the cluster threshold, stopping said progressively filling the group of matrix elements and placing said first nucleotide string and said second nucleotide string in two different clusters, said output diagonal being a diagonal of the matrix comprising the matrix element corresponding to the last column and the last row of the matrix.

### BRIEF DESCRIPTION OF THE ANNEXED DRAWINGS

These and other features and advantages of the present invention will be made apparent by the following description of some exemplary and non-limitative embodiments thereof. For its better intelligibility, the following description should be read making reference to the attached drawings, wherein:

FIG. 1 depicts exemplary reference DNA strands and corresponding exemplary DNA strand replicas;

FIG. 2 illustrates an exemplary clustering of the DNA strand replicas of FIG. 1;

FIGS. 3A-3D illustrate an example of how an edited distance between two nucleotide strings is calculated using the Wagner-Fischer algorithm;

FIG. 4 illustrates in terms of very simplified functional blocks a synthetic DNA storage system wherein concepts according to the embodiments of the present invention can be applied;

FIG. 5 shows an example of possible elements of a write module and a read module of the synthetic DNA storage system of FIG. 4;

FIG. 6 is a flow chart illustrating main operations performed by the read module of the synthetic DNA storage system of FIG. 4 for deciding if two nucleotide strings have to be placed in a same or in different clusters according to an embodiment of the present invention;

FIGS. 7A-7G illustrate an example of how matrix elements are filled column-by-column for deciding if two nucleotide strings have to be placed in a same or in different clusters according to an embodiment of the present invention;

FIGS. 8A-8F illustrate an example of how matrix elements are filled column-by-column for deciding if two nucleotide strings have to be placed in a same or in different clusters according to an embodiment of the present invention;

FIGS. 9A-9H illustrate an example of how matrix elements are filled antidiagonal-by-antidiagonal for deciding if two nucleotide strings have to be placed in a same or in different clusters according to an embodiment of the present invention;

FIGS. 10A-10B show a matrix provided with boundary diagonals according to an embodiment of the present invention;

FIG. 11 show a matrix provided with boundary diagonals according to another embodiment of the present invention;

FIG. 12 shows a matrix provided with boundary diagonals according to a still further embodiment of the present invention.

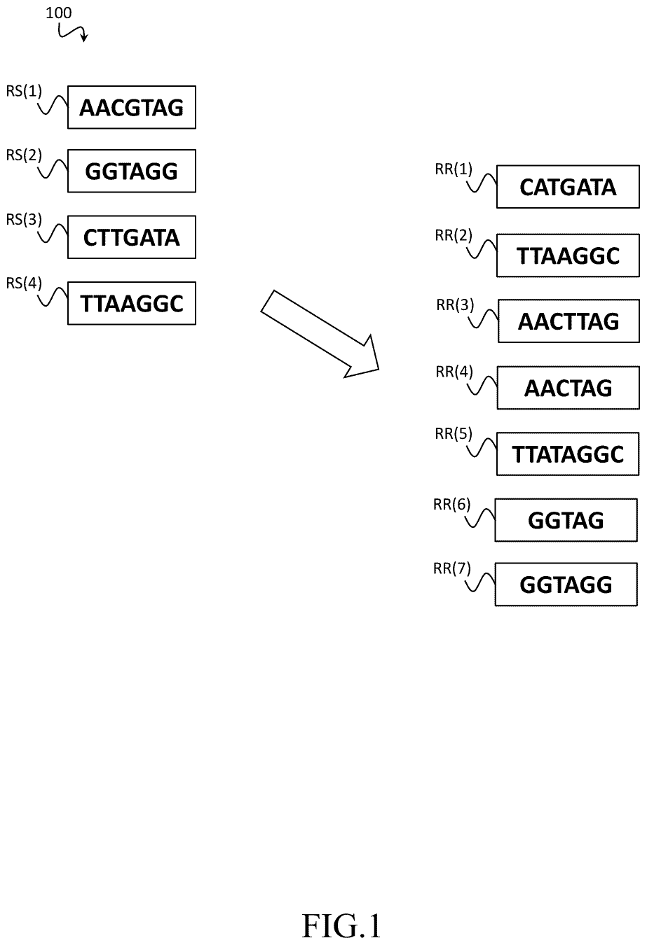

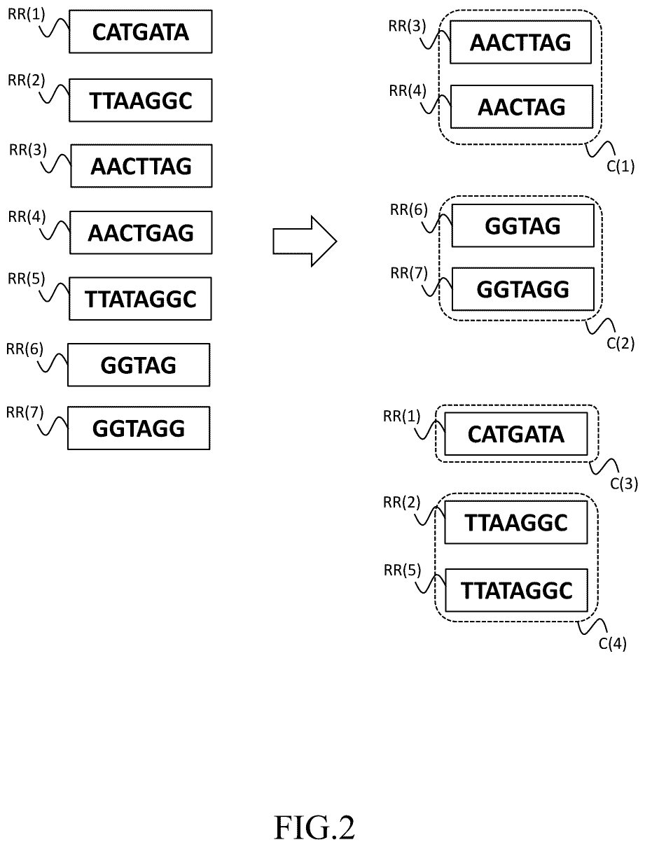

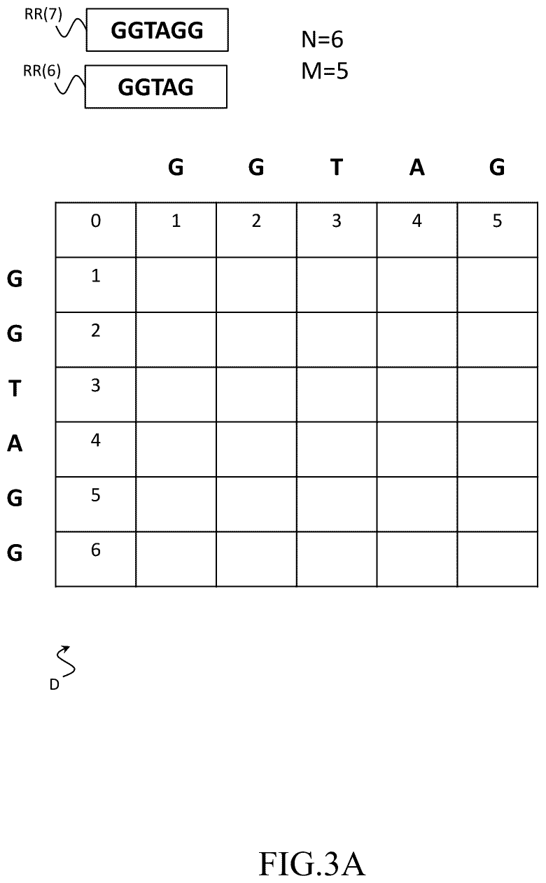

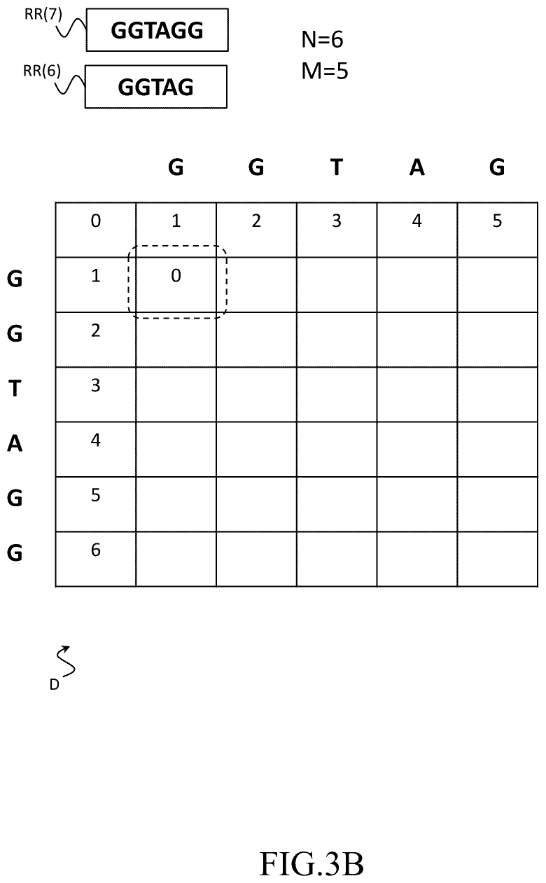

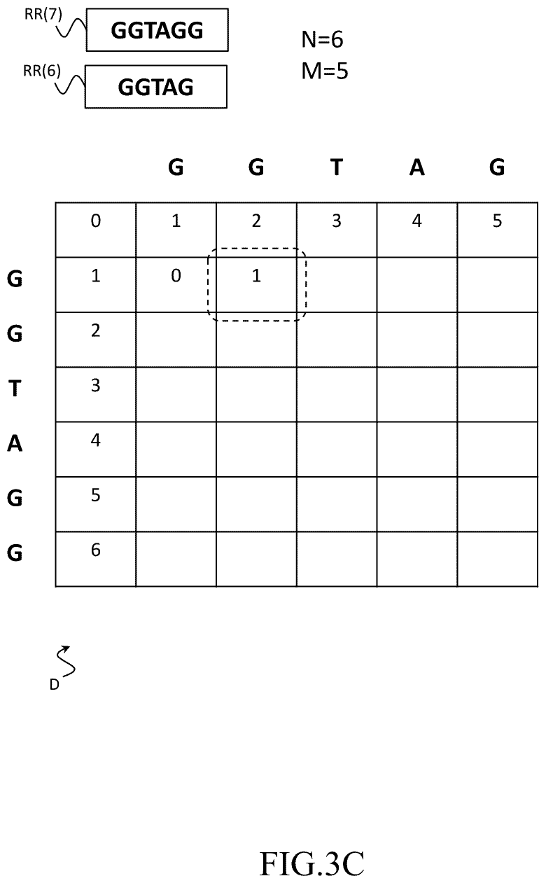

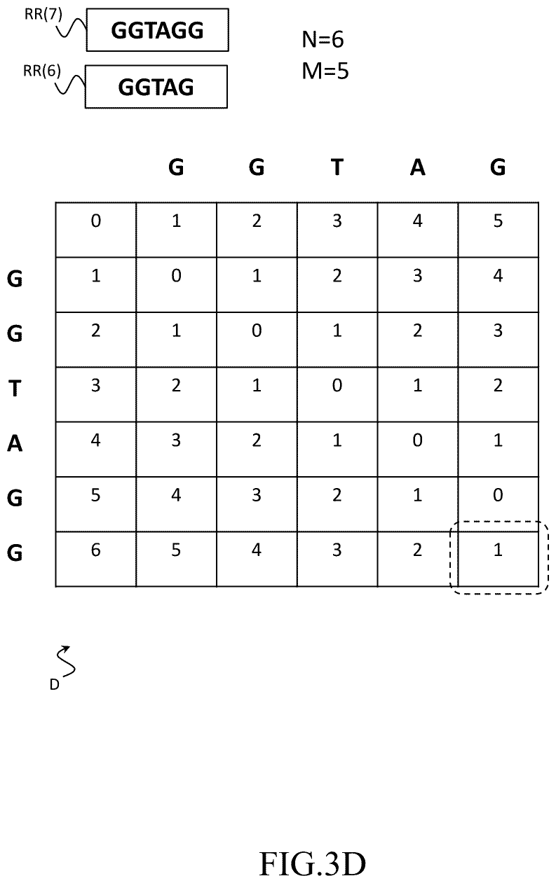

### DETAILED DESCRIPTION OF PREFERRED EMBODIMENTS OF THE INVENTION

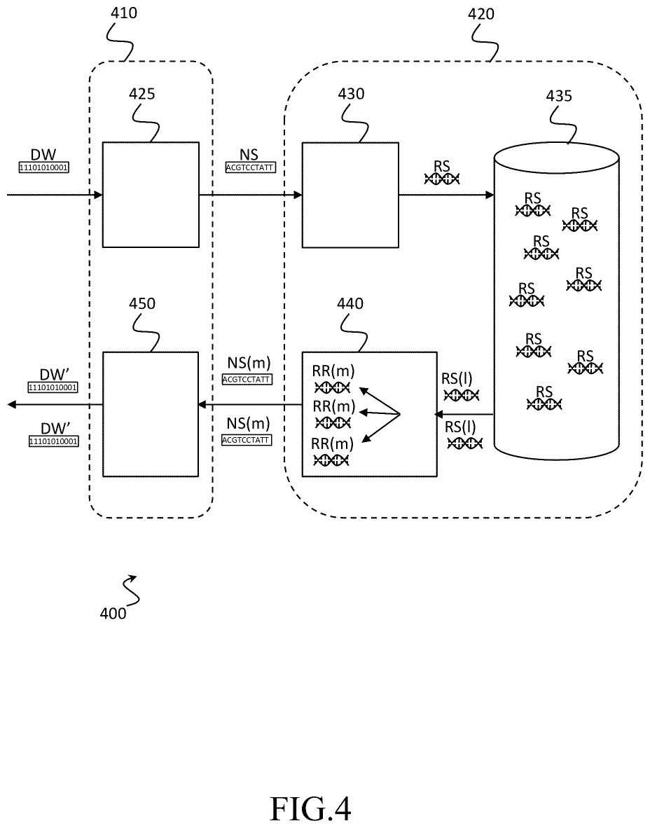

FIG. 4 illustrates in terms of very simplified functional blocks a synthetic DNA storage system 400 wherein concepts according to the embodiments of the present invention can be applied.

The system 400 comprises two main sections, and namely a digital processing section 410 adapted to perform operations on strings of symbols, and a DNA processing section 420 adapted to perform operations on DNA strands.

The digital processing section 410 comprises a write module 425 configured to receive digital data in form of digital words DW (e.g., comprising sequences of 0 and 1 symbols) and to accordingly generate nucleotide strings NS comprising sequences of symbols belonging to the alphabet {A, C, G, T} through encoding/mapping/randomization techniques known in the art.

The DNA processing section 420 comprises a synthesizer module 430 configured to receive nucleotide strings NS from the digital processing section 410 and to accordingly synthesize artificial DNA strands RS whose nucleotides match the received nucleotide strings NS, for example through one of the known procedures based on oligonucleotide synthesis.

The DNA processing section 420 further comprises a DNA storage module 435 configured to store the artificial DNA strands RS generated by the synthesizer module 430. As a non-limitative example, the DNA storage module 435 comprises arrays of liquid vessels filled with liquid suitable to preserve the stored artificial DNA strands RS generated by the synthesizer module 430.

The DNA processing section 420 further comprises a DNA sequencer module 440 configured to retrieve from the DNA storage module 435 a selected group of stored artificial DNA strands RS (reference DNA strands RS(l) (l=1, 2, . . . )) to be read and amplify the retrieved reference DNA strands RS(l) through one of the known DNA sequencing procedures for generating a plurality of DNA strands replicas RR(m) (m=1, 2, . . . ) of the retrieved reference DNA strands RS(1).

The DNA sequencer module 440 is further configured to output for each DNA strands replica RR(m) generated through sequencing the corresponding nucleotide string NS(m).

The digital processing section 410 further comprises a read module 450 configured to receive the nucleotide strings NS(m) of the DNA strands replicas RR(m) generated by the DNA sequencer module 440, cluster the nucleotide strings NS(m) into corresponding clusters C(p), process the clusters C(p) according to known retrieval algorithms (e.g., a consensus-finding algorithm) to retrieve from the received nucleotide strings NS(m) the actual nucleotide strings of the selected group of reference DNA strands RS(l), and decode them to obtain corresponding output digital words DW′.

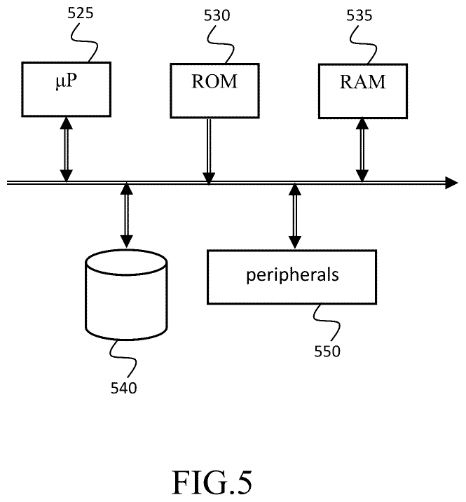

Passing now to FIG. 5, each of the above-described modules of the digital processing section 410 (write module 425, read module 450) comprises several units that are connected among them through a bus structure 510 at one or more levels (with an architecture that is suitably scaled according to the type of the module). Particularly, a microprocessor (μP) 525, or more, provides a logic capability of the modules 425, 450, a non-volatile memory (ROM) 530 stores basic code for a bootstrap of the modules 425, 450 and a volatile memory (RAM) 535 is used as a working memory by the microprocessor 525. The modules 425, 450 are provided with a mass-memory 540 for storing programs and data. Moreover, the modules 425, 450 comprise a number of controllers for peripherals, or Input/Output (I/O) units, 550.

A single system comprising the units depicted in FIG. 5 may be also provided to carry out both the function of the write module 425 and of the read module 450.

According to an embodiment of the present invention, the read module 450 is configured to decide if two nucleotide strings NS(1), NS(2) received by the DNA sequencer module 440 have to be placed in a same or in different clusters C(p) exploiting a modified version of the Wagner-Fischer algorithm which does not require to necessarily calculate the entire edit distance ED between the two nucleotide strings NS(1), NS(2), i.e., without having to necessarily calculate all the possible edit distances ED(i,j) to fill all the matrix elements d[i,j] of the matrix D.

Applicant has observed that when the matrix D is filled according to the Wagner-Fischer algorithm, the values of the edit distances ED(i,j) calculated across the matrix elements d[i,j] of the diagonals of the matrix D have a non-decreasing behavior, i.e., ED(i,j)≤ED(i+1,j+1). Since the edit distance ED between a first nucleotide string NS(1) comprising N symbols and a second nucleotide string NS(2) comprising M symbols is the edit distance ED(i=N, j=M) corresponding to the last matrix element d[i=N,j=M] of the matrix D, also the edit distances ED(i,j) corresponding to the diagonal of the matrix D comprising said last matrix element d[i=N,j=M]—i.e., the diagonal comprising the matrix elements d[N−k, M−k] (k=min(N,M), min(N,M)−1, . . . , 1, 0)—exhibit a non-decreasing behavior. Said diagonal comprising the last matrix element d[N, M] is hereinafter referred to as “output diagonal” OD.

The modified version of the Wagner-Fischer algorithm according to the embodiments of the present invention is based on deciding if two nucleotide strings NS(1), NS(2) have to be placed in a same or in different clusters C(p) based on the edit distance ED as compared to the cluster threshold TH rather than the exact value of the edit distance ED. If the edit distance ED is lower than the cluster threshold TH, the nucleotide strings NS(1), NS(2) are considered to be sufficiently similar to each other to be placed in a same cluster C(p), otherwise they are placed in different clusters C(p). Since the edit distances ED(i,j) corresponding to the matrix elements d[i,j] of the output diagonal OD have a non-decreasing behavior as i and j increase, the algorithm according to the embodiments of the present invention is configured to interrupt the process of calculating edit distances ED(i,j) already before the calculation of the last edit distance ED(i=N, j=M) once an edit distance ED(i,j) corresponding to a matrix element d[i,j] of the output diagonal OD different from the last matrix element d[N, M] has been calculated to be already not lower than the cluster threshold TH. Indeed, if the edit distance ED(i,j) corresponding to a matrix element d[i,j] of the output diagonal OD different from the last matrix element d[N, M] already reached (or exceeded) the cluster threshold TH, the edit distance ED(N, M) corresponding to the last matrix element d[i=N,j=M] will be not lower than the threshold TH because of the non-decreasing behavior of the edit distances ED(i,j) in the output diagonal OD as i and j increase. Therefore, thanks to the algorithm according to the embodiments of the present invention, it is advantageously possible to reduce the number of computations required to decide if two nucleotide strings NS(1), NS(2) have to be placed in a same or in different clusters C(p).

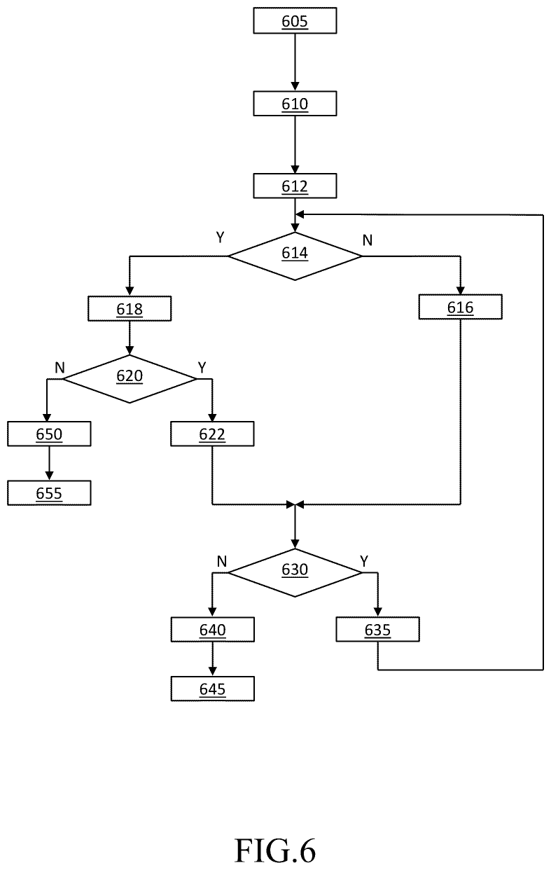

FIG. 6 is a flow chart illustrating the main operations performed by the read module 450 for deciding if two nucleotide strings NS(1)=“x(1) x(2) . . . x(i) . . . X(N)”, NS(2)=“y(1) y(2) . . . y(j) . . . y(M)” received by the DNA sequencer module 440 have to be placed in a same or in different clusters C(p) exploiting a modified version of the Wagner-Fischer algorithm according to an embodiment of the present invention.

According to an embodiment of the present invention, the read module 450 arranges a matrix D having N+1 rows and M+1 columns, (block 605).

According to an embodiment of the present invention, the read module 450 initializes the elements d[0, j] (j=0 to M) of the row r(0) of the matrix D to values ED(0,j)=j, respectively, and initializes the elements d[i, 0] (i=0 to N) of the column c(0) of the matrix D to values ED(i,0)=i, respectively (block 610).

According to an embodiment of the present invention, the read module 450 sets the column index j to 1, so that the next operations are directed to the calculation of the edit distances ED(i,1) corresponding to the matrix elements d[i, 1] of the first column c(j=1) (block 612).

According to an embodiment of the present invention, the read module 450 checks if the column c(j) comprises a matrix element d[i,j] of the output diagonal OD (block 614).

According to an embodiment of the present invention, if the column c(j) does not comprise a matrix element d[i,j] of the output diagonal OD (exit branch N of block 614), the read module 450 calculates the edit distances ED(i,j) (i=1 to N) corresponding to the column c(j) (block 616) as previously described, i.e.:

$$ED ( i , j ) = min ( \begin{matrix}ED ( i - 1 , j - 1 ) + s \\ ED ( i - 1 , j ) + 1 \\ ED ( i , j - 1 ) + 1\end{matrix} )$$

$$wherein :$$

$$s = \{ \begin{matrix}0 & if x ( i ) = y ( j ) \\ 1 & if x ( i ) \neq y ( j )\end{matrix}$$

According to an embodiment of the present invention, if instead the column c(j) comprises a matrix element d[i,j] of the output diagonal OD (exit branch Y of block 614), the read module 450 calculates the edit distances ED(i,j) (i=1 to j+N−M) corresponding to the column c(j) up to the matrix element d[j+N−M,j] belonging to the output diagonal OD (block 618).

According to an embodiment of the present invention, the read module 450 checks if the edit distance E(j+N−M,j) is lower than the cluster threshold TH (block 620).

According to an embodiment of the present invention, if the edit distance E(j+N−M,j) is lower than the cluster threshold TH (exit branch Y of block 620), the read module 450 calculates the remaining edit distances ED(i,j) (i=j+N−M+1 to N) of the column c(j) (block 622).

According to an embodiment of the present invention, once all the edit distances ED(i,j) of the j-th column c(j) have been calculated (block 616 or block 622), the read module 450 checks if said column c(j) is the last column c(M) of the matrix D (block 630).

According to an embodiment of the present invention, if the column c(j) is not the last one of the matrix D (exit branch Y of block 630), the read module 450 increments the column index j by one (block 635), ad reiterates the previously described operations for the new column c(j) (return to block 614).

According to an embodiment of the present invention, if the column c(j) is the last one of the matrix (exit branch N of block 630), it means that all the edit distances ED(i,j) have been calculated, and the read module 450 assesses that the edit distance ED(N, M) corresponding to the last matrix element d[i=N,j=M], and corresponding to the actual edit distance between the two nucleotide strings NS(1), NS(2), is lower than the cluster threshold TH (block 640). Therefore, according to an embodiment of the present invention, the read module 450 places the two nucleotide strings NS(1), NS(2) into a same cluster C(p) because it assessed that their similarity level is sufficiently high (block 645).

Returning back to block 620, according to an embodiment of the present invention, if the edit distance E(j+N−M,j) is not lower than the cluster threshold TH (exit branch N of block 620), the read module 450 already assesses (block 650) that the actual edit distance ED(N, M) between the two nucleotide strings NS(1), NS(2) will be in any case not lower than the cluster threshold TH even without calculating it because its value will be at least equal to the edit distance E(j+N−M,j). Therefore, according to an embodiment of the present invention, the read module 450 places the two nucleotide strings NS(1), NS(2) into a different cluster C(p) because it assessed that their similarity level is not sufficient (block 655).

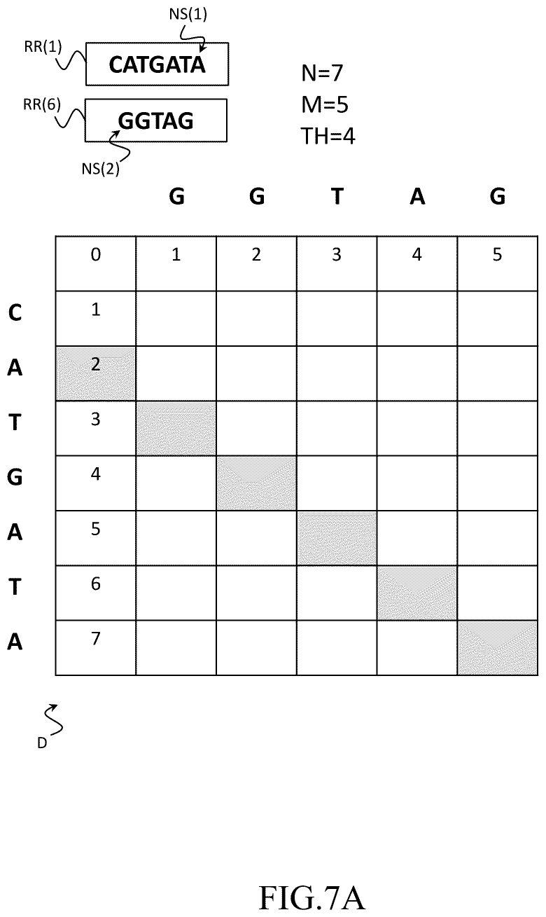

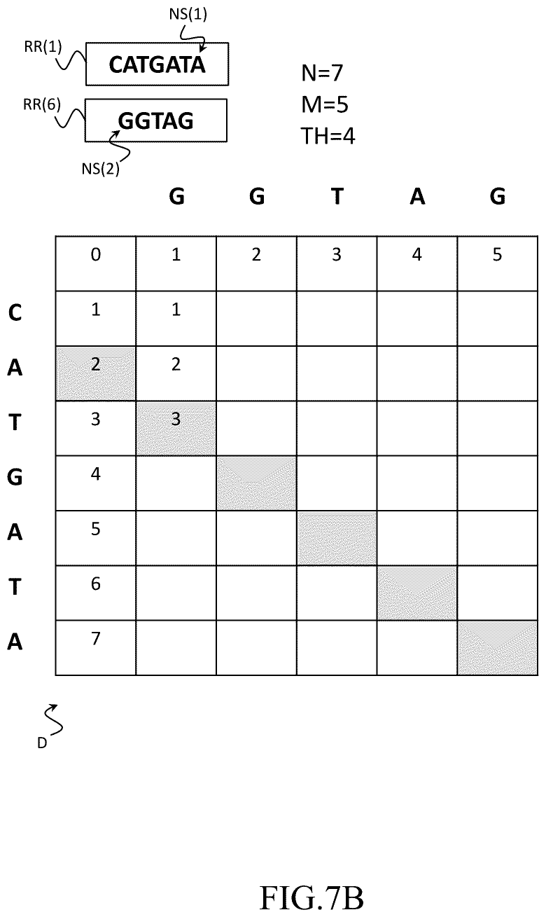

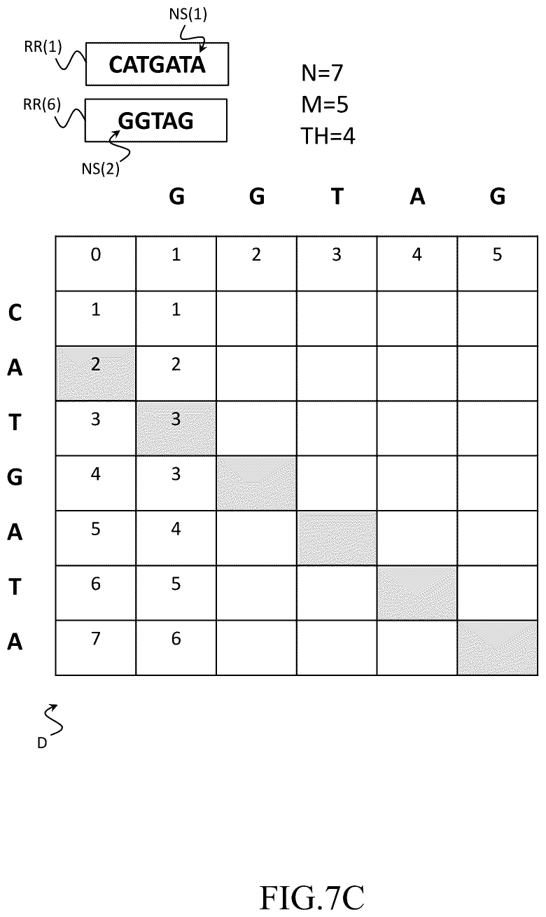

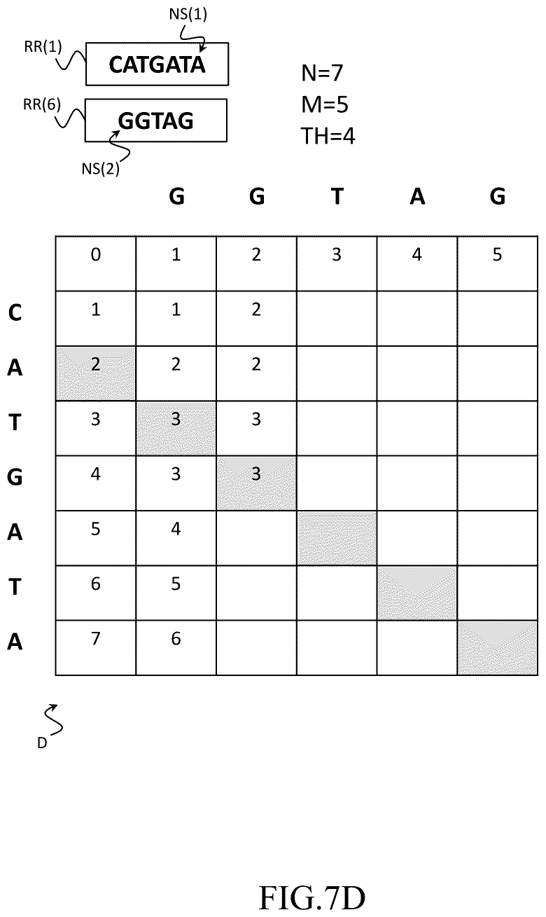

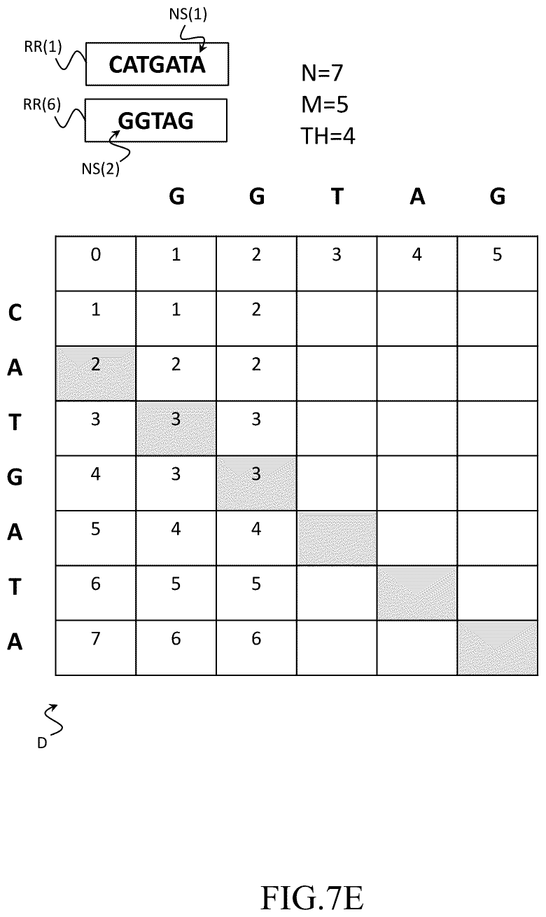

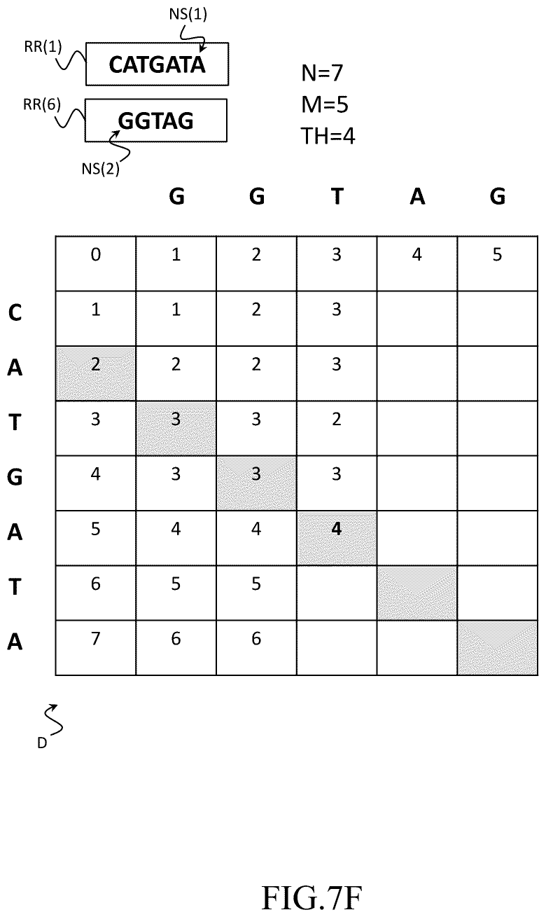

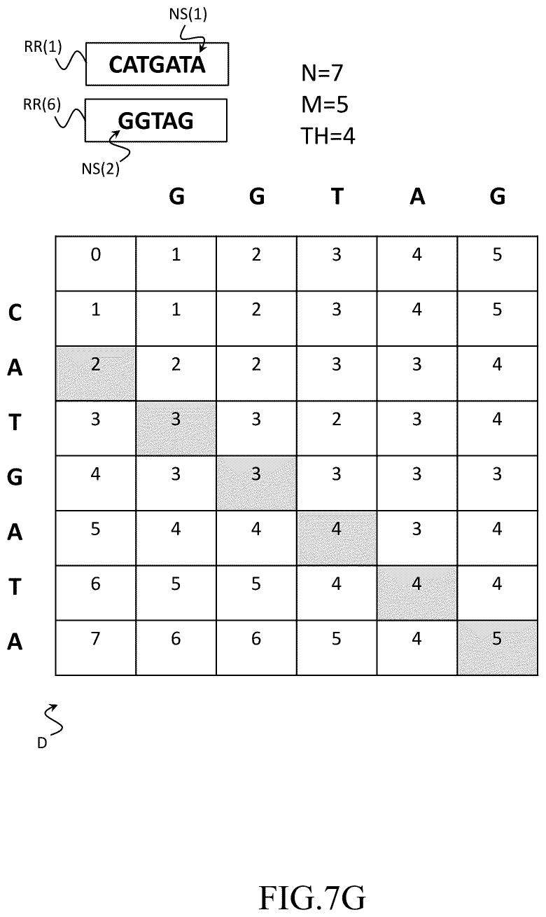

With reference to FIGS. 7A-7G, an example will be now described of how the matrix elements d[i,j] of the matrix D are filled when the operations described in the flow chart of FIG. 6 are performed by the read module 450 for deciding if the nucleotide string NS(1)=x(1) x(2) . . . x(i) . . . X(N=7)”=“CATGATA” of the exemplary DNA strand replica RR(1) and the nucleotide string NS(2)=x(1) x(2) . . . x(i) . . . X(M=5)” =“GGTAG” of the exemplary DNA strand replica RR(6) have to be placed in a same or in different clusters C(p) according to an embodiment of the present invention.

In the considered example, the cluster threshold TH is equal to 4.

Moreover, in the considered example, the matrix D has N+1=8 rows and M+1=6 columns; the elements d[0, j] (j=0 to M=5) of the row r(0) of the matrix D are initialized to values ED(0,j)=j, respectively, and the elements d[i, 0] (i=0 to N=7) of the column c(0) of the matrix D are initialized to values ED(i,0)=i, respectively (see FIG. 7A).

In the exemplary case illustrated in the FIGS. 7A-7G, the output diagonal OD comprises the matrix elements d[2,0], d[3,1], d[4,2], d[5,3], d[6,4], d[7,5], highlighted in the figures with a grey shading.

According to an embodiment of the invention, the read module 450 starts to fill the column c(1) of the matrix D by calculating the edit distances ED(i,1) (i=1, 2, . . . ) until the edit distance ED(3,1) corresponding to the matrix element d[3,1] belonging to the output diagonal OD (see FIG. 7B). Since in the example at issue the edit distance ED(3,1)=3 is lower than the cluster threshold TH=4, according to an embodiment of the present invention, the read module 450 calculates the remaining edit distances ED(i,1) (i=4, 5, . . . ) corresponding to the column c(1) of the matrix D (see FIG. 7C).

According to an embodiment of the invention, the read module 450 passes to the next column c(2) of the matrix D by calculating the edit distances ED(i,2) (i=1, 2, . . . ) until the edit distance ED(4,2) corresponding to the matrix element d[4,2] belonging to the output diagonal OD (see FIG. 7D). Since in the example at issue also the edit distance ED(4,2)=3 is lower than the cluster threshold TH=4, according to an embodiment of the present invention, the read module 450 calculates the remaining edit distances ED(i,2) (i=5, 6, . . . ) corresponding to the column c(2) of the matrix D (see FIG. 7E).

According to an embodiment of the invention, the read module 450 passes to the next column c(3) of the matrix D by calculating the edit distances ED(i,3) (i=1, 2, . . . ) until the edit distance ED(5,3) corresponding to the matrix element d[5,3] belonging to the output diagonal OD (see FIG. 7F). Since in the example at issue the edit distance ED(5,3)=4 is not lower than the cluster threshold TH=4, according to an embodiment of the present invention, the read module 450 stops the calculation of further edit distances ED(i,j) for further matrix elements d[i,j], since it has already assessed that the actual edit distance ED(N,M) between the nucleotide strings NS(1) and NS(2) cannot be lower than the cluster threshold TH, and therefore the nucleotide strings NS(1) and NS(2) have to be placed in different clusters C(p).

Compared to the known solutions, requiring the explicit calculation of the actual edit distance ED(N,M), the solution according to the embodiments of the invention allows to cluster nucleotide strings requiring a potentially lower number of calculations of edit distances ED(i,j). In the example at issue, in which the actual edit distance ED(N=7, M=5) is equal to 5, the solution according to the embodiments of the invention allowed to save the calculation of sixteen edit distances ED(i,j) (compare FIG. 7F and FIG. 7G).

Although the flow chart illustrated in FIG. 6 and the corresponding example illustrated in FIGS. 7A-7G provide for calculating the edit distances ED(i,j) by proceeding column-by-column, the concepts of the present invention can be directly applied to different way of scanning the matrix D.

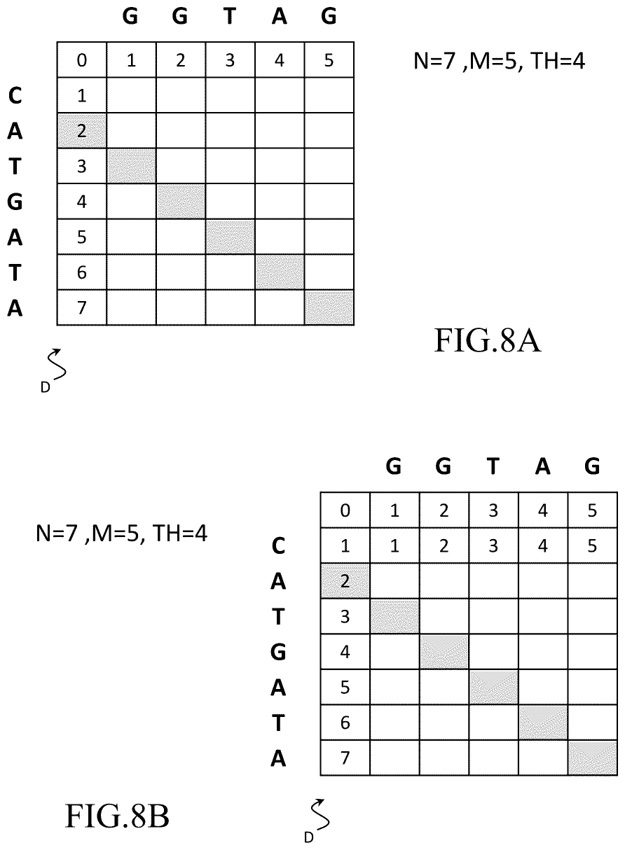

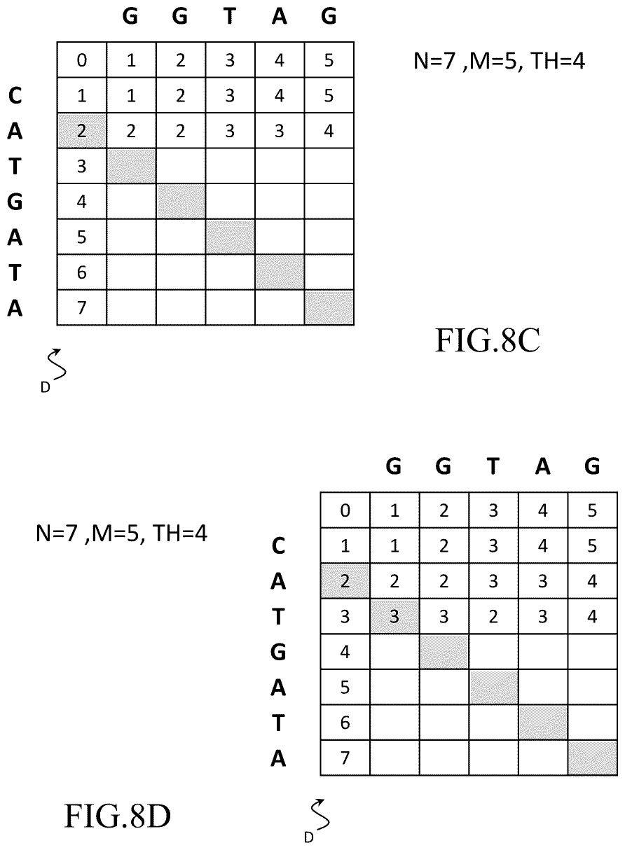

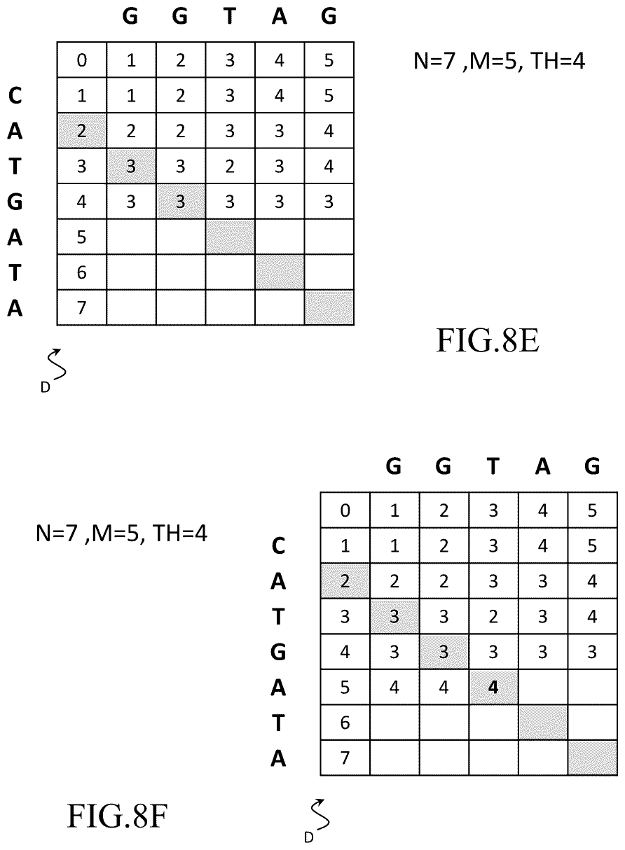

FIGS. 8A-8F show an example in which the concepts of the present invention are applied to a procedure in which the edit distances ED(i,j) are calculated by proceeding row-by-row, using the same pair of nucleotide strings NS(1), NS(2) already used for the example illustrated in FIGS. 7A-7G.

In the considered example, the cluster threshold TH is still equal to 4.

According to an embodiment of the present invention, starting from an initialization of the matrix equal to the one used for the previous example, i.e., with the elements d[0, j] (j=0 to M=5) of the row r(0) of the matrix D that are initialized to values ED(0,j)=j, respectively, and the elements d[i, 0] (i=0 to N=7) of the column c(0) of the matrix D that are initialized to values ED(i,0)=i, respectively (see FIG. 8A), the read module 450 starts to fill the row r(1) of the matrix D by calculating the edit distances ED(1,j) (j=1, 2, . . . ).

According to an embodiment of the present invention, since in the example at issue the row r(1) does not comprise matrix elements d[i,j] belonging to the output diagonal OD, all the edit distances ED(1,j) (j=1 to 5) of the row r(1) are directly calculated (see FIG. 8B).

Then, according to an embodiment of the present invention, the read module 450 passes to the next row r(2) of the matrix D and calculates the edit distances ED(2,j) (j=1, . . . , 5) (see FIG. 8C).

According to an embodiment of the present invention, the read module 450 passes to the next row r(3) of the matrix D and calculates the edit distance ED(3,1) corresponding to the matrix element d[3,1] belonging to the output diagonal OD. Since in the example at issue the edit distance ED(3,1)=3 is lower than the cluster threshold TH=4, according to an embodiment of the present invention, the read module 450 calculates the remaining edit distances ED(3,j) (j=2, 3, . . . ) corresponding to the row r(3) of the matrix D (see FIG. 8D).

According to an embodiment of the invention, the read module 450 passes to the next row r(4) of the matrix D by calculating the edit distances ED(4,j) (j=1, 2) until the edit distance ED(4,2) corresponding to the matrix element d[4,2] belonging to the output diagonal OD. Since in the example at issue also the edit distance ED(4,2)=3 is lower than the cluster threshold TH=4, according to an embodiment of the present invention, the read module 450 calculates the remaining edit distances ED(4,j) (j=3, 4, . . . ) corresponding to the row r(4) of the matrix D (see FIG. 8E).

According to an embodiment of the invention, the read module 450 passes to the next row r(5) of the matrix D by calculating the edit distances ED(5,j) (j=1, 2, . . . ) until the edit distance ED(5,3) corresponding to the matrix element d[5,3] belonging to the output diagonal OD. Since in the example at issue the edit distance ED(5,3)=4 is not lower than the cluster threshold TH=4, according to an embodiment of the present invention, the read module 450 stops the calculation of further edit distances ED(i,j) for further matrix elements d[i,j] (see FIG. 8F), since it has already assessed that the actual edit distance ED(N,M) between the nucleotide strings NS(1) and NS(2) cannot be lower than the cluster threshold TH, and therefore the nucleotide strings NS(1) and NS(2) have to be placed in different clusters C(p).

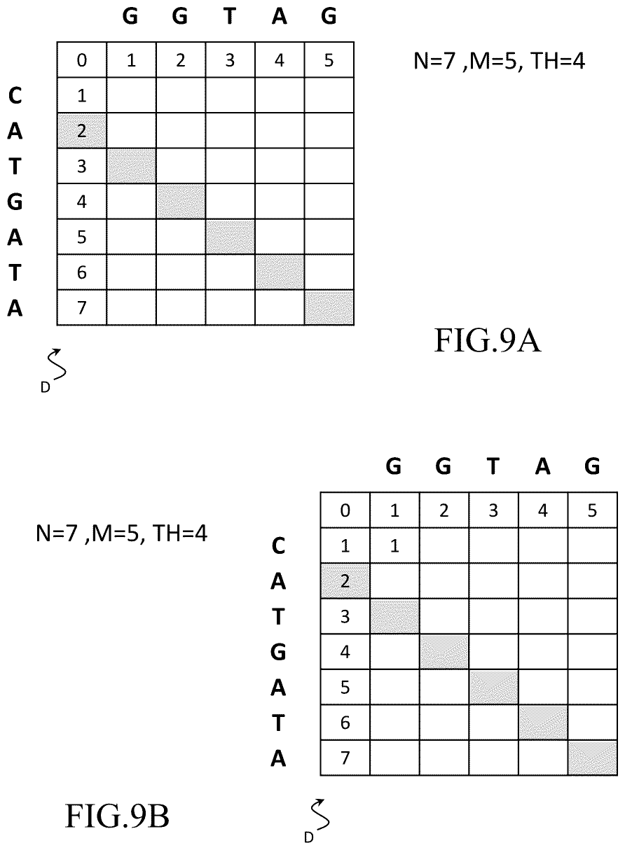

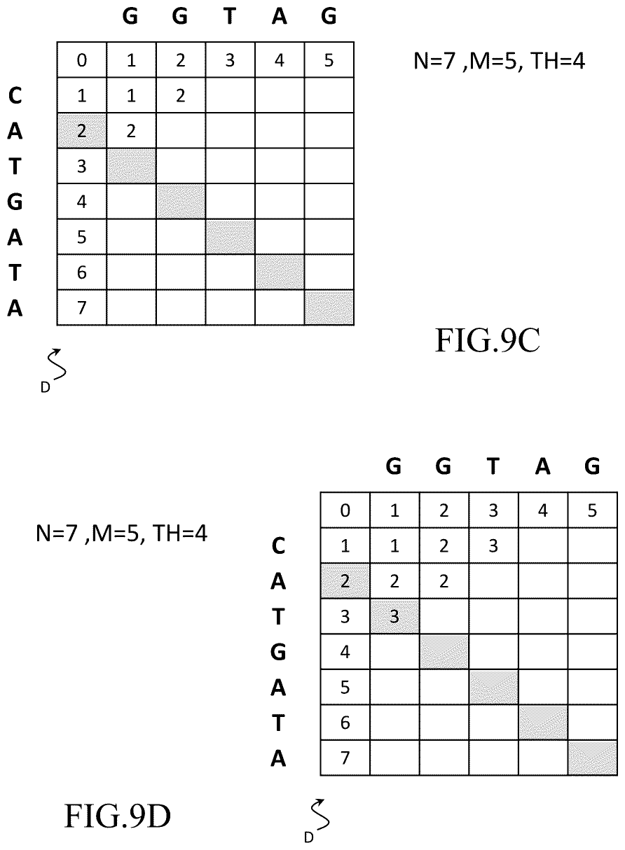

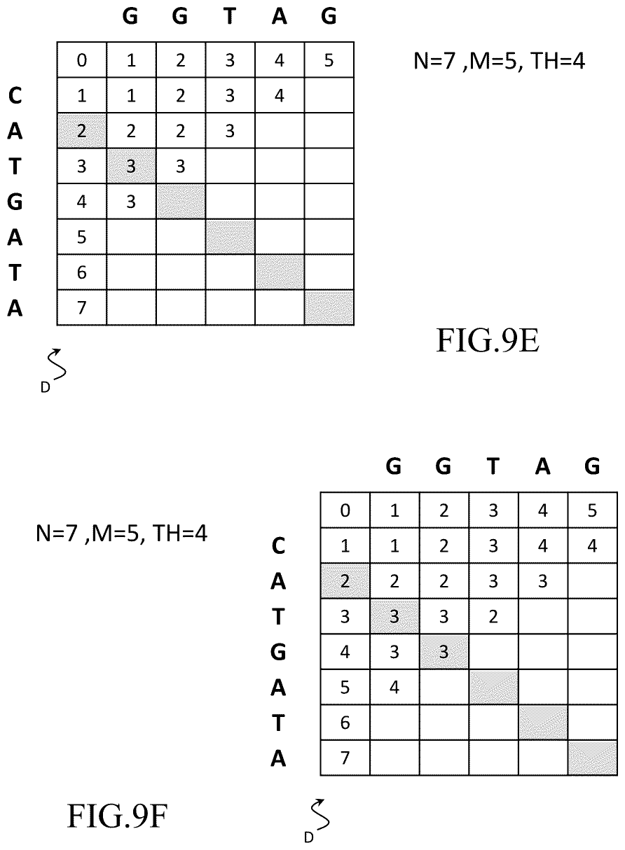

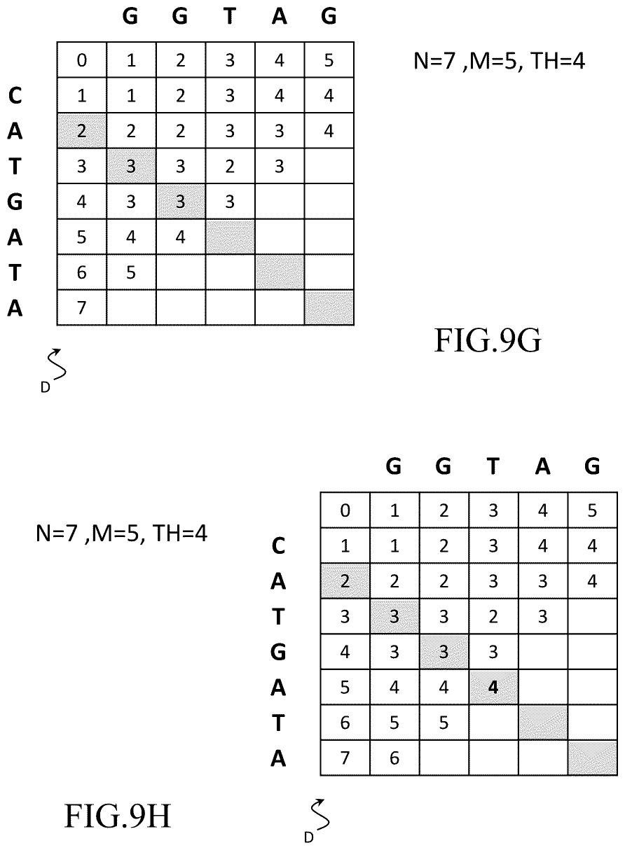

FIGS. 9A-9H show an example in which the concepts of the present invention are applied to a procedure in which the edit distances ED(i,j) are calculated by proceeding antidiagonal-by-antidiagonal, using the same pair of nucleotide strings NS(1), NS(2) already used for the example illustrated in FIGS. 7A-7G.

In the considered example, the cluster threshold TH is still equal to 4.

According to an embodiment of the present invention, starting from an initialization of the matrix equal to the one used for the previous example, i.e., with the elements d[0, j] (j=0 to M=5) of the row r(0) of the matrix D that are initialized to values ED(0,j)=j, respectively, and the elements d[i, 0] (i=0 to N=7) of the column c(0) of the matrix D that are initialized to values ED(i,0)=i, respectively (see FIG. 9A), the read module 450 starts to fill the antidiagonal comprising the matrix element d[1,1] by calculating the edit distance ED(1,1) (see FIG. 9B).

According to an embodiment of the present invention, the read module 450 passes to the next antidiagonal of the matrix D, i.e., the one comprising the matrix elements d[2, 1], d[1, 2]. According to an embodiment of the present invention, since in the example at issue this antidiagonal does not comprise matrix elements d[i,j] belonging to the output diagonal OD, all the edit distances ED(2, 1), ED(1, 2) of the antidiagonal are directly calculated (see FIG. 9C).

Then, according to an embodiment of the present invention, the read module 450 passes to the next antidiagonal of the matrix D, i.e., the one comprising the matrix elements d[3, 1], d[2, 2], d[1, 3], and calculates the edit distance ED(3,1) corresponding to the matrix element d[3,1] belonging to the output diagonal OD. Since in the example at issue the edit distance ED(3,1)=3 is lower than the cluster threshold TH=4, according to an embodiment of the present invention, the read module 450 calculates the remaining edit distances ED(2, 2), ED(1, 3) of the antidiagonal (see FIG. 9D).

According to an embodiment of the present invention, the read module 450 passes to the next antidiagonal of the matrix D, i.e., the one comprising the matrix elements d[4, 1], d[3, 2], d[2, 3], d[1, 4]. According to an embodiment of the present invention, since in the example at issue this antidiagonal does not comprise matrix elements d[i,j] belonging to the output diagonal OD, all the edit distances ED(i, j) of the antidiagonal are directly calculated (see FIG. 9E).

Then, according to an embodiment of the present invention, the read module 450 passes to the next antidiagonal of the matrix D, i.e., the one comprising the matrix elements d[5, 1], d[4, 2], d[3, 3], d[2, 4], d[1, 5] and calculates the edit distances ED(i,j) of the antidiagonal until the edit distance ED(4, 2) corresponding to the matrix element d[4,2] belonging to the output diagonal OD. Since in the example at issue the edit distance ED(4,2)=3 is lower than the cluster threshold TH=4, according to an embodiment of the present invention, the read module 450 calculates the remaining edit distances ED(i, j) of the antidiagonal (see FIG. 9F).

According to an embodiment of the present invention, the read module 450 passes to the next antidiagonal of the matrix D, i.e., the one comprising the matrix elements d[6, 1], d[5, 2], d[4, 3], d[3, 4], d[2, 5]. According to an embodiment of the present invention, since in the example at issue this antidiagonal does not comprise matrix elements d[i,j] belonging to the output diagonal OD, all the edit distances ED(i, j) of the antidiagonal are directly calculated (see FIG. 9G).

Then, according to an embodiment of the present invention, the read module 450 passes to the next antidiagonal of the matrix D, i.e., the one comprising the matrix elements d[7, 1], d[6, 2], d[5, 3], d[4, 4], d[3, 5] and calculates the edit distances ED(i,j) of the antidiagonal until the edit distance ED(5, 3) corresponding to the matrix element d[5,3] belonging to the output diagonal OD. Since in the example at issue the edit distance ED(5,3)=4 is not lower than the cluster threshold TH=4, according to an embodiment of the present invention, the read module 450 stops the calculation of further edit distances ED(i,j) for further matrix elements d[i,j] (see FIG. 9H), since it has already assessed that the actual edit distance ED(N,M) between the nucleotide strings NS(1) and NS(2) cannot be lower than the cluster threshold TH, and therefore the nucleotide strings NS(1) and NS(2) have to be placed in different clusters C(p).

Although in the example illustrated in FIGS. 9A-9H the edit distances ED(i,j) of each antidiagonal are calculated by proceeding from “bottom-left” to “top right”, the concepts of the present invention can be directly applied in case the edit distances ED(i,j) of each antidiagonal are calculated by proceeding from “top right” to “bottom-left”.

The embodiments of the invention described above provide for the potential calculation of the edit distances ED(i,j) corresponding to a group GR of matrix elements d[i,j] comprising all the matrix elements d[i,j] of the matrix D, with the advantageous possibility that the procedure is subjected to an early stop if an edit distance ED(i,j) corresponding to a matrix element d[i,j] belonging to the output diagonal OD has been assessed to be not lower that the cluster threshold TH.

However, the concepts of the present invention can be applied in case the group GR of matrix elements d[i,j] to be considered for being processed according to the procedures described above is only a subset of all the matrix elements d[i,j] of the matrix D, provided that said group GR comprise the matrix elements d[i,j] corresponding to the output diagonal OD. In other words, an early stop of the procedure may be still carried out if an edit distance ED(i,j) corresponding to a matrix element d[i,j] of the (reduced) group GR belonging to the output diagonal OD has been assessed to be not lower that the cluster threshold TH.

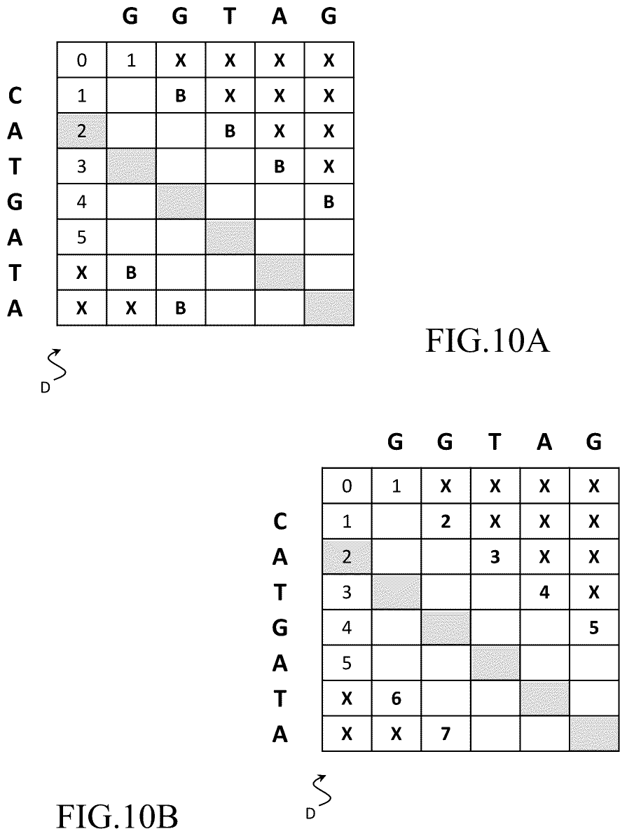

According to an embodiment of the invention illustrated in FIG. 10A, the group GR of matrix elements d[i,j] comprise matrix elements d[i,j] that are comprised between two diagonals of the matrix D (hereinafter referred to as “boundary diagonals BD” and whose matrix elements d[i,j] are depicted in FIG. 10A with reference B) that are different from the output diagonal OD, wherein the output diagonal OD falls between the boundary diagonals BD. The matrix elements d[i,j] that do not belong to the group GR (depicted in FIG. 10A with reference X) are skipped, and not considered for the calculation of the edit distances ED(i,j). Thanks to this modification—that is based on the known Ukkonen algorithm—the number of operations necessary for assessing if two nucleotide strings NS(1) and NS(2) have to be placed in a same cluster C(p) or in different clusters C(p), is potentially further reduced. It is underlined that by using a reduced group GR of matrix elements d[i,j] of the matrix like the one illustrated in FIG. 10A, the resulting values calculated for each matrix element d[i,j] may be only indicative of the actual edit distances ED(i,j), because of the approximation error introduced by skipping the matrix elements X. However, since the purpose of the algorithms according to the embodiments of the present invention is to assess if two nucleotide strings NS(1) and NS(2) have to be placed in a same cluster C(p) or in different clusters C(p), and not to exactly calculate the actual value of their edit distance ED(N,M), said approximation can be considered acceptable.

According to an embodiment of the present invention, the boundary diagonals BD comprise a first boundary diagonal BD1 that is displaced “toward right” with respect to the output diagonal OD by a corresponding number ND of matrix elements d[i,j] higher than IN-MI, and a second boundary diagonal BD2 that is displaced “toward left” with respect to the output diagonal OD by said number ND of matrix elements d[i,j]. In other words, according to this embodiment of the present invention, if matrix element d[i,j] is a matrix element belonging to the output diagonal OD, the matrix element d[i,j+ND] is a matrix element of the first boundary diagonal BD1 (provided that it is included in the matrix D and it is not a matrix element d[i,j] of the initialized row r(0) or column c(0)), and the matrix element d[i,j−ND] is a matrix element of the second boundary diagonal BD2 (provided that it is included in the matrix D and it is not a matrix element d[i,j] of the initialized row r(0) or column c(0)). In the example illustrated in FIG. 10A, the number ND is equal to 3, so that the boundary diagonal BD1 comprises 4 matrix elements d[i,j] and the boundary diagonal BD2 comprises 2 matrix elements d[i,j].

According to an embodiment of the present invention, the values stored in the matrix elements d[i,j] of the boundary diagonals BD1, BD2 are set to the maximum possible edit value ED(i,j) that may be reached in a (i+1)×(j+1) matrix D. By making reference to the example illustrated in FIG. 10B, the four matrix elements d[i,j] of the boundary diagonal BD1 are set to 2, 3, 4, 5, respectively, and the two matrix elements d[i,j] of the boundary diagonal BD2 are set to 6, 7, respectively.

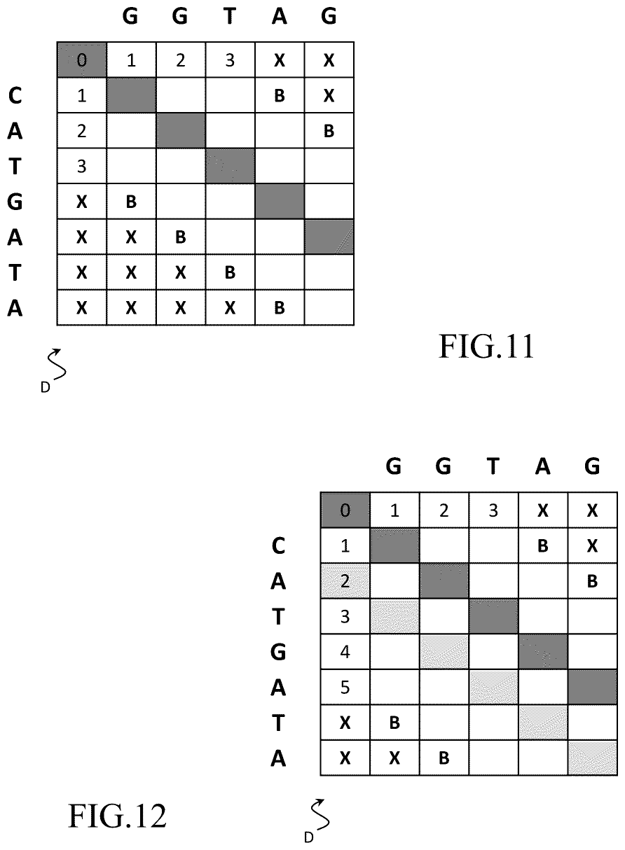

According to an embodiment of the present invention illustrated in FIG. 11, the boundary diagonals BD comprise a first boundary diagonal BD1′ that is displaced “toward right” with respect to the main diagonal MD of the matrix D (whose matrix elements d[i,j] are highlighted in the figures with a dark grey shading) by a corresponding number ND′ of matrix elements d[i,j], and a second boundary diagonal BD2′ that is displaced “toward left” with respect to the main diagonal MD by said number ND′ of matrix elements d[i,j]. In other words, according to this embodiment of the present invention, if matrix element d[i,j] is a matrix element belonging to the main diagonal MD, the matrix element d[i,j+ND′] is a matrix element of the first boundary diagonal BD1′ (provided that it is included in the matrix D and it is not a matrix element d[i,j] of the initialized row r(0) or column c(0)), and the matrix element d[i,j−ND′] is a matrix element of the second boundary diagonal BD2′ (provided that it is included in the matrix D and it is not a matrix element d[i,j] of the initialized row r(0) or column c(0)). In the example illustrated in FIG. 11, the number ND′ is equal to 3, so that the boundary diagonal BD1′ comprises 2 matrix elements d[i,j] and the boundary diagonal BD2′ comprises 4 matrix elements d[i,j].

According to an embodiment of the present invention illustrated in FIG. 12, mixed solutions are also contemplated, in which the boundary diagonals BD comprise a first boundary diagonal BD1″ that is displaced “toward right” with respect to the output diagonal OD (or to the main diagonal MD) of the matrix D by a corresponding number ND″ of matrix elements d[i,j], and a second boundary diagonal BD2″ that is displaced “toward left” with respect to the main diagonal MD (or to the output diagonal OD) by a corresponding number ND″ of matrix elements d[i,j]. In the example illustrated in FIG. 12, the numbers ND″ and ND′″ are both equal to 3, so that the boundary diagonal BD1″ comprises 2 matrix elements d[i,j] and the boundary diagonal BD2″ comprises 2 matrix elements d[i,j].

It is underlined that while in the embodiments of the invention described above the edit distances ED(i,j) are calculated one at a time, the concepts of the present invention can be directly applied also to cases in which the edit distances ED(i,j) corresponding to a set ST comprising more than one matrix element d[i,j] (e.g., to an entire row or column of the matrix D) are calculated in a single step, for example exploiting a bit-parallel algorithm, such as the known Myers bit-parallel algorithm (Myers, G. (1999), “A fast bit-vector algorithm for approximate string matching based on dynamic programming”, Journal of the ACM (JACM), 46(3), 395-415, DOI: 10.1007/BFb0030777) or the known Hyyro bit-parallel algorithm (Hyyro, H. (2003), “A Bit-Vector Algorithm for Computing Levenshtein and Damerau Edit Distances”, NA, 10, 29-39, see also https://researchportal.tuni.fi/en/publications/a-bit-vector-algorithm-for-computing-levenshtein-and-damerau-edit-2).

In other words, an early stop of the procedure may be still carried out if an edit distance ED(i,j) corresponding to a matrix element d[i,j] of said set ST that belongs also to the output diagonal OD has been assessed to be not lower that the cluster threshold TH.

Naturally, in order to satisfy local and specific requirements, a person skilled in the art may apply to the present invention as described above many logical and/or physical modifications and alterations. More specifically, although the present invention has been described with a certain degree of particularity with reference to preferred embodiments thereof, it should be understood that various omissions, substitutions and changes in the form and details as well as other embodiments are possible. In particular, different embodiments of the invention may even be practiced without the specific details set forth in the preceding description for providing a more thorough understanding thereof; on the contrary, well-known features may have been omitted or simplified in order not to encumber the description with unnecessary details. Moreover, it is expressly intended that specific elements and/or method steps described in connection with any disclosed embodiment of the invention may be incorporated in any other embodiment.

What is claimed is:

1. A method for reading a group of synthetic DNA strands, comprising:
amplifying the DNA strands of the group so as to generate, for each DNA strand of the group, at least a corresponding DNA strand replica, each DNA strand replica corresponding to a respective nucleotide string comprising a sequence of nucleotides of the DNA strand replica;
clustering the nucleotide strings of the generated DNA strand replicas in respective clusters so that each cluster comprises nucleotide strings having edit distances among them that are lower than a cluster threshold;
obtaining a reading of at least one DNA strand of the group based on the nucleotide strings comprised in a at least one among said clusters,
wherein said clustering the nucleotide strings in respective clusters comprises:
for each pair of a first nucleotide string and a second nucleotide string, carrying out the following sequence of operations:
arranging a matrix of matrix elements, the matrix comprising a respective row for each nucleotide in the first nucleotide string, and a respective column for each nucleotide in the second nucleotide string, each matrix element which corresponds to the row corresponding to a selected nucleotide in the first nucleotide string and to the column corresponding to a further selected nucleotide in the second nucleotide string being configured to store a calculated edit value indicative of an edit distance between a prefix of the first nucleotide string ending with said selected nucleotide and a prefix of the second nucleotide string ending with said selected further nucleotide;
starting from the matrix element which corresponds to the row corresponding to the first nucleotide of the first nucleotide string and to the column corresponding to the first nucleotide of the second nucleotide string, progressively filling a group of matrix elements by storing calculated edit values indicative of edit distances corresponding to said matrix elements;
if the edit value calculated for a matrix element belonging to an output diagonal of the matrix is not lower than the cluster threshold, stopping said progressively filling the group of matrix elements and placing said first nucleotide string and said second nucleotide string in two different clusters, said output diagonal being a diagonal of the matrix comprising the matrix element corresponding to the last column and the last row of the matrix.

2. The method of claim 1, further comprising:
if the edit value calculated for the matrix element corresponding to the last column and the last row of the matrix is lower than the cluster threshold, placing said first nucleotide string and said second nucleotide string in a same cluster.

3. The method of claim 1, wherein said filling a group of matrix elements by storing calculated edit values comprises storing into a selected matrix element corresponding to a selected row and to a selected column a calculated edit value that is calculated based on a comparison between:
the nucleotide in the first nucleotide string corresponding to said selected row, and
the nucleotide in the second nucleotide string corresponding to said selected column.

4. The method of claim 1, wherein said filling the matrix elements by storing calculated edit values comprises storing into a selected matrix element corresponding to a selected row and to a selected column a calculated edit value that is calculated based on a comparison among already calculated edit values stored in matrix elements adjacent to said selected matrix element.

5. The method of claim 1, wherein said filling the matrix elements by storing calculated edit values comprises storing into a selected matrix element corresponding to a selected row and to a selected column a calculated edit value that is calculated according to the following procedure:
setting a parameter to zero if the nucleotide in the first nucleotide string corresponding to said selected row is equal to the nucleotide in the second nucleotide string corresponding to said selected column;
setting said parameter to one if the nucleotide in the first nucleotide string corresponding to said selected row is different from the nucleotide in the second nucleotide string corresponding to said selected column;
setting the edit value as the minimum among a), b), c):
a) the value of said parameter plus the calculated edit value stored in the matrix element corresponding to:
the row of the matrix adjacent to and preceding the selected row, and
the column of the matrix adjacent to and preceding the selected column;
b) one plus the calculated edit value stored in the matrix element corresponding to:
the row of the matrix adjacent to and preceding the selected row, and
the selected column;
c) one plus the calculated edit value stored in the matrix element corresponding to:
the selected row, and
the column of the matrix adjacent to and preceding the selected column.

6. The method of claim 1, wherein:
the rows of the matrix are ordered according to the order of the nucleotides in the first nucleotide string;
the columns of the matrix are ordered according to the order of the nucleotides in the second nucleotide string.

7. The method of claim 1, wherein said matrix further comprises:
an initialization row adjacent to and preceding the row corresponding to the first nucleotide of the first nucleotide string;
an initialization column adjacent to and preceding the column corresponding to the second nucleotide of the second nucleotide string,
wherein the method further comprises:
before said progressively filling the group of matrix elements starting from the matrix element which corresponds to the row corresponding to the first nucleotide of the first nucleotide string and to the column corresponding to the first nucleotide of the second nucleotide string, carrying out the following operations:
initializing the matrix elements of the initialization row by storing edit values having an increasing value, starting from zero, and progressively increasing by one at each adjacent subsequent matrix element of the initialization row;
initializing the matrix values of the initialization column by storing edit values having an increasing value, starting from zero, and progressively increasing by one at each adjacent subsequent matrix element of the initialization column.

8. The method of claim 1, wherein said progressively filling the group of matrix elements by storing calculated edit values comprises calculating and storing edit values by proceeding according to a row-by-row pattern starting from said matrix element which corresponds to the row corresponding to the first nucleotide of the first nucleotide string and to the column corresponding to the first nucleotide of the second nucleotide string.

9. The method of claim 1, wherein said progressively filling the group of matrix elements by storing calculated edit values comprises calculating and storing edit values by proceeding according to a column-by-column pattern starting from said matrix element which corresponds to the row corresponding to the first nucleotide of the first nucleotide string and to the column corresponding to the first nucleotide of the second nucleotide string.

10. The method of claim 1, wherein said progressively filling the group of matrix elements by storing calculated edit values comprises calculating and storing edit values by proceeding according to an antidiagonal-by-antidiagonal pattern starting from said matrix element which corresponds to the row corresponding to the first nucleotide of the first nucleotide string and to the column corresponding to the first nucleotide of the second nucleotide string.

11. The method of claim 1, wherein said group of matrix elements comprise all the matrix elements of the matrix.

12. The method of claim 1, wherein said group of matrix elements comprise the matrix elements corresponding to the output diagonal.

13. The method of claim 12, wherein said group of matrix elements comprise matrix elements comprised between two diagonals of the matrix different from the output diagonal, the output diagonal falling between said two diagonals.

14. The method of claim 13, wherein said two diagonals of the matrix different from the output diagonal comprise:
a first diagonal that is displaced with respect to the output diagonal by a corresponding number of matrix elements;
a second diagonal that is displaced with respect to the output diagonal by said number of matrix elements.

15. The method of claim 13, wherein said two diagonals of the matrix different from the output diagonal comprise:
a first diagonal that is displaced with respect to the main diagonal of the matrix by a corresponding number of matrix elements;
a second diagonal that is displaced with respect to the main diagonal of the matrix by said number of matrix elements.

16. The method of claim 13, wherein said two diagonals of the matrix different from the output diagonal comprise:
a first diagonal that is displaced with respect to the main diagonal of the matrix by a corresponding number of matrix elements;
a second diagonal that is displaced with respect to the output diagonal by said number of matrix elements.

17. A method for clustering a group of synthetic DNA strands, each one corresponding to a respective nucleotide string comprising a sequence of nucleotides of the DNA strand, in respective clusters so that each cluster comprises nucleotide strings having edit distances among them that are lower than a cluster threshold, the method comprising:
for each pair of a first nucleotide string and a second nucleotide string, carrying out the following sequence of operations:
arranging a matrix of matrix elements, the matrix comprising a respective row for each nucleotide in the first nucleotide string, and a respective column for each nucleotide in the second nucleotide string, each matrix element which corresponds to the row corresponding to a selected nucleotide in the first nucleotide string and to the column corresponding to a further selected nucleotide in the second nucleotide string being configured to store a calculated edit value indicative of an edit distance between a prefix of the first nucleotide string ending with said selected nucleotide and a prefix of the second nucleotide string ending with said selected further nucleotide;
starting from the matrix element which corresponds to the row corresponding to the first nucleotide of the first nucleotide string and to the column corresponding to the first nucleotide of the second nucleotide string, progressively filling a group of matrix elements by storing calculated edit values indicative of edit distances corresponding to said matrix elements;
if the edit value calculated for a matrix element belonging to an output diagonal of the matrix is not lower than the cluster threshold, stopping said progressively filling the group of matrix elements and placing said first nucleotide string and said second nucleotide string in two different clusters, said output diagonal being a diagonal of the matrix comprising the matrix element corresponding to the last column and the last row of the matrix.

18. The method of claim 17, further comprising:
if the edit value calculated for the matrix element corresponding to the last column and the last row of the matrix is lower than the cluster threshold, placing said first nucleotide string and said second nucleotide string in a same cluster.

19. The method of claim 17, wherein said filling the matrix elements by storing calculated edit values comprises storing into a selected matrix element corresponding to a selected row and to a selected column a calculated edit value that is calculated according to the following procedure:
setting a parameter to zero if the nucleotide in the first nucleotide string corresponding to said selected row is equal to the nucleotide in the second nucleotide string corresponding to said selected column;
setting said parameter to one if the nucleotide in the first nucleotide string corresponding to said selected row is different from the nucleotide in the second nucleotide string corresponding to said selected column;
setting the edit value as the minimum among a), b), c):
a) the value of said parameter plus the calculated edit value stored in the matrix element corresponding to:
the row of the matrix adjacent to and preceding the selected row, and
the column of the matrix adjacent to and preceding the selected column;
b) one plus the calculated edit value stored in the matrix element corresponding to:
the row of the matrix adjacent to and preceding the selected row, and
the selected column;
c) one plus the calculated edit value stored in the matrix element corresponding to:
the selected row, and
the column of the matrix adjacent to and preceding the selected column.

20. The method of claim 17, wherein:
the rows of the matrix are ordered according to the order of the nucleotides in the first nucleotide string;
the columns of the matrix are ordered according to the order of the nucleotides in the second nucleotide string.

21. The method of claim 17, wherein said matrix further comprises:
an initialization row adjacent to and preceding the row corresponding to the first nucleotide of the first nucleotide string;
an initialization column adjacent to and preceding the column corresponding to the second nucleotide of the second nucleotide string,
wherein the method further comprises:
before said progressively filling the group of matrix elements starting from the matrix element which corresponds to the row corresponding to the first nucleotide of the first nucleotide string and to the column corresponding to the first nucleotide of the second nucleotide string, carrying out the following operations:
initializing the matrix elements of the initialization row by storing edit values having an increasing value, starting from zero, and progressively increasing by one at each adjacent subsequent matrix element of the initialization row;
initializing the matrix values of the initialization column by storing edit values having an increasing value, starting from zero, and progressively increasing by one at each adjacent subsequent matrix element of the initialization column.

22. The method of claim 17, wherein said progressively filling the group of matrix elements by storing calculated edit values comprises calculating and storing edit values by proceeding according to a row-by-row pattern starting from said matrix element which corresponds to the row corresponding to the first nucleotide of the first nucleotide string and to the column corresponding to the first nucleotide of the second nucleotide string.

23. The method of claim 17, wherein said progressively filling the group of matrix elements by storing calculated edit values comprises calculating and storing edit values by proceeding according to a column-by-column pattern starting from said matrix element which corresponds to the row corresponding to the first nucleotide of the first nucleotide string and to the column corresponding to the first nucleotide of the second nucleotide string.

24. The method of claim 17, wherein said progressively filling the group of matrix elements by storing calculated edit values comprises calculating and storing edit values by proceeding according to an antidiagonal-by-antidiagonal pattern starting from said matrix element which corresponds to the row corresponding to the first nucleotide of the first nucleotide string and to the column corresponding to the first nucleotide of the second nucleotide string.

25. The method of claim 17, wherein said group of matrix elements comprise the matrix elements corresponding to the output diagonal.

26. The method of claim 25, wherein said group of matrix elements comprise matrix elements comprised between two diagonals of the matrix different from the output diagonal, the output diagonal falling between said two diagonals.

27. A synthetic DNA storage system, comprising:
a DNA storage module configured to store synthetic DNA strands;
a sequencer module configured to amplify a group of stored synthetic DNA strands so as to generate, for each DNA strand of the group, at least a corresponding DNA strand replica, each DNA strand replica corresponding to a respective nucleotide string comprising a sequence of nucleotides of the DNA strand replica;
a reading module configured to:
cluster the nucleotide strings of the generated DNA strand replicas in respective clusters so that each cluster comprises nucleotide strings having edit distances among them that are lower than a cluster threshold, and
obtain a reading of at least one DNA strand of the group based on the nucleotide strings comprised in a at least one among said clusters,
wherein said reading module is configured to cluster the nucleotide strings in respective clusters by carrying out for each pair of a first nucleotide string and a second nucleotide string the following sequence of operations:
arranging a matrix of matrix elements, the matrix comprising a respective row for each nucleotide in the first nucleotide string, and a respective column for each nucleotide in the second nucleotide string, each matrix element which corresponds to the row corresponding to a selected nucleotide in the first nucleotide string and to the column corresponding to a further selected nucleotide in the second nucleotide string being configured to store a calculated edit value indicative of an edit distance between a prefix of the first nucleotide string ending with said selected nucleotide and a prefix of the second nucleotide string ending with said selected further nucleotide;
starting from the matrix element which corresponds to the row corresponding to the first nucleotide of the first nucleotide string and to the column corresponding to the first nucleotide of the second nucleotide string, progressively filling a group of matrix elements by storing calculated edit values indicative of edit distances corresponding to said matrix elements;
if the edit value calculated for a matrix element belonging to an output diagonal of the matrix is not lower than the cluster threshold, stopping said progressively filling the group of matrix elements and placing said first nucleotide string and said second nucleotide string in two different clusters, said output diagonal being a diagonal of the matrix comprising the matrix element corresponding to the last column and the last row of the matrix.
